(19) United States
(12) Patent Application Publication
(10) Pub. No.: US 2025/0369003 A1
(43) Pub. Date: Dec. 4, 2025

(54) METHOD FOR CONSTRUCTING LACTIC ACID-PRODUCING STRAINS, LACTIC ACID-PRODUCING STRAINS, AND USE THEREOF

(71) Applicant: Shanghai Jiao Tong University, Shanghai (CN)

(72) Inventors: Ping Xu, Shanghai (CN); Jiongqin Liu, Shanghai (CN); Fei Tao, Shanghai (CN)

(73) Assignee: Shanghai Jiao Tong University, Shanghai (CN)

(21) Appl. No.: 19/301,160

(22) Filed: Aug. 15, 2025

Related U.S. Application Data
(63) Continuation-in-part of application No. PCT/CN2024/074884, filed on Jan. 31, 2024.

(30) Foreign Application Priority Data
Feb. 17, 2023 (CN) 202310134446.2

(51) Int. Cl. C12N 15/74; C12N 9/02; C12N 9/04; C12N 9/10; C12N 9/12; C12N 15/52; C12N 15/75; C12P 7/56

(52) U.S. Cl. CPC C12N 15/74; C12N 9/0006; C12N 9/0008; C12N 9/1029; C12N 9/1205; C12N 15/52; C12N 15/75; C12P 7/56; C12Y 101/01001; C12Y 101/01027; C12Y 101/01028; C12Y 102/0101; C12Y 203/01008; C12Y 203/01054; C12Y 207/01011; C12Y 207/0104

(57) ABSTRACT

Provided are a method for constructing a lactic acid-producing strain, a lactic acid-producing strain and use thereof. The method for constructing lactic acid-producing strains is characterized by genetically engineering a starting strain to increase lactic acid production, wherein the engineering includes: 1) introducing a lactic acid synthesis pathway; 2) optimizing the lactic acid synthesis pathway; and 3) inhibiting by-product synthesis pathways.

### RELATED APPLICATIONS

This application is a continuation-in-part (CIP) application claiming benefit of PCT/CN2024/074884 filed on Jan. 31, 2024, which claims priority to Chinese Patent Application No. 202310134446.2 filed on Feb. 17, 2023, the disclosures of which are incorporated herein in their entirety by reference.

Reference to an Electronic Sequence Listing A Sequence Listing is submitted herewith in accordance with the requirements of 37 CFR § 1.821-1.825 and WIPO Standard ST.26. The Sequence Listing is filed as a separate electronic file under the file name “389405.00002 Sequence Listing.xml,” created on Aug. 12, 2025, and is incorporated herein by reference in its entirety. The Sequence Listing contains 81 sequences and is 84,159 bytes in size.

### FIELD OF THE INVENTION

The present invention relates to the fields of genetic engineering, metabolic engineering, fermentation engineering, enzyme engineering and synthetic biology, and more particularly to a method for constructing a lactic acid-producing strain, a lactic acid-producing strain and use thereof.

### DESCRIPTION OF THE PRIOR ART

Lactic acid is a naturally occurring, high-value-added, important three-carbon platform chemical, and according to chirality, can be divided into the two types: L-lactic acid and D-lactic acid, which are produced from catalysis of pyruvic acid by L-lactate dehydrogenase and D-lactate dehydrogenase, respectively, in the presence of cofactors. L-lactic acid and D-lactic acid can be widely used in food, medicine, cosmetics, and petrochemical industries. Most importantly, they can be used as precursors for use in the production of biodegradable plastic polylactic acid. Changing the ratio of the two chiral precursors in the polymer can improve the heat resistance, hydrolysis resistance, and mechanical properties of polylactic acid. Therefore, optically pure L-lactic acid and D-lactic acid appear to be very important, and their market demands are very large.

High-temperature fermentation has the following advantages: (1) it can reduce the cooling cost of large-scale exothermic fermentation, and this advantage becomes increasingly obvious with the increase in fermentation scale; (2) it can decrease the risk of contamination by mesophilic microorganisms, because these microorganisms cannot survive at high temperatures; (3) it facilitates industrial biological processes for simultaneous saccharification and fermentation and reduce the cost of enzymes used in these processes because the most suitable reaction temperatures for these enzymes are all relatively high; (4) it can make reactions unfavorable to mesophilic microorganisms thermodynamically feasible; (5) it provides fermentation media with good properties, such as low viscosity, fast substrate diffusion and good substrate solubility; and (6) it is conductive to the production, collection, and extraction of volatile products, because this can reduce substrate inhibition, substrate toxicity and other influence. Therefore, selecting thermophilic hosts for high-temperature fermentation has great value in industrial applications.

Therefore, those skilled in the art are devoted to developing a strategy for constructing efficient lactic acid-producing strains. The efficient lactic acid-producing strains obtained using this strategy can be used to produce lactic acid by high-temperature fermentation. This is conducive to industrialized production of lactic acid and is conducive to promoting rapid development of the lactic acid industry.

### SUMMARY OF THE INVENTION

In order to solve the above described technical problems, the object of the present invention is to provide a method for constructing a lactic acid-producing strain, a lactic acid-producing strain and use thereof.

Specifically, the present invention provides:
(1) a method for constructing a lactic acid-producing strain, characterized by genetically engineering a starting strain to increase lactic acid production, wherein the engineering comprises: 1) introducing a lactic acid synthesis pathway; 2) optimizing the lactic acid synthesis pathway; and 3) inhibiting by-product synthesis pathways;
(2) the method according to (1), wherein the starting strain is a lactic acid- and/or pyruvic acid-producing strain; preferably, the starting strain is able to produce lactic acid and/or pyruvic acid at a temperature of 37° C. to 70° C.;
(3) the method according to (2), wherein the starting strain includes microorganisms of genus Bacillus and Geobacillus;
(4) the method according to (3), wherein the starting strain includes Bacillus lichenformis, Bacillus coagulans, Bacillus smithii, Bacillus pallidus and Geobacillus thermoglucosidasius;
(5) the method according to (1), wherein introducing the lactic acid synthesis pathway comprises introducing an L-lactate dehydrogenase gene L-ldh or a D-lactate dehydrogenase gene D-ldh into the starting strain;
(6) the method according to (1), wherein optimizing the lactic acid synthesis pathway comprises introducing, into the starting strain, coding sequences of one or more genes selected from: a lactate dehydrogenase gene ldh, a 6-phosphofructokinase gene pfk and a pyruvate kinase gene pyk, wherein the lactate dehydrogenase gene ldh is an L-lactate dehydrogenase gene L-ldh or a D-lactate dehydrogenase gene D-ldh;
(7) the method according to (5) or (6), wherein the introduction into the starting strain comprises: integration into a genome of the starting strain or expression in a plasmid form in the starting strain; preferably, the introduction comprises introducing single-copy or multi-copy the coding sequences of the genes; preferably, the coding sequences of the genes are introduced as individual single-gene expression fragments or as tandem expression fragments of the coding sequences of the genes;
(8) the method according to (1), wherein inhibiting the by-product synthesis pathways comprises knocking out or down one or more coding genes in the starting strain selected from: a lactate dehydrogenase gene ldh, a formate acetyltransferase gene pflB, an acetaldehyde dehydrogenase gene acdh, an aldehyde-alcohol dehydrogenase gene aadh and a phosphate acetyltransferase gene pta, wherein the lactate dehydrogenase gene ldh is an L-lactate dehydrogenase gene L-ldh or a D-lactate dehydrogenase gene D-ldh;
(9) the method according to (1), wherein the lactic acid is L-lactic acid or D-lactic acid;
(10) the method according to (5) or (6), wherein the D-lactate dehydrogenase gene ldh encodes an amino acid sequence as shown in SEQ ID No. 9 or SEQ ID No. 10, or encodes an amino acid sequence exhibiting 30% or higher identity to the amino acid sequence as shown in SEQ ID No. 9 or SEQ ID No. 10;
(11) the method according to (5) or (6), wherein the L-lactate dehydrogenase gene L-ldh is derived from Bacillus coagulans and/or Geobacillus thermoglucosidasius, the 6-phosphofructokinase gene pfk from Bacillus coagulans and the pyruvate kinase gene pyk from Bacillus coagulans;
(12) a lactic acid-producing strain, characterized by being constructed in accordance with the method according to any of (1) to (11);
(13) the strain according to (12), wherein the strain is Geobacillus thermoglucosidasius with deposition Nos. CCTCC M 20221822, CCTCC M 20221823, CCTCC M 20221824 and CCTCC M 20221825;
(14) use of the strain according to (12) or (13) in lactic acid production;
(15) the use of (14), wherein the lactic acid is L-lactic acid or D-lactic acid;
(16) the use of (14), wherein the lactic acid has a titer of at least 80 g L⁻¹, and a yield of at least 80%; preferably, the lactic acid has a chiral purity of at least 99.04%;
(17) a method for producing lactic acid, characterized by comprising steps of:
1) providing the lactic acid-producing strain according to (12) or (13);
2) culturing the strain at 37° C. to 70° C. for 6 hours to 15 hours, thereby providing a seed; and
3) subjecting the seed to fermentation culture at 37° C. to 70° C. in a presence of a carbon source, thereby obtaining the lactic acid;
(18) the method according to (17), wherein the carbon source is selected from one or more of glucose, xylose, sucrose, glycerol, arabinose and mannitol; preferably, the lactic acid is L-lactic acid or D-lactic acid;
(19) the method according to (17), wherein in step 3), an inoculum volume of the seed has an OD_{620 nm} value of 0.2 to 0.8; preferably, an initial concentration of the carbon source is 40 g/L to 100 g/L; preferably, the fermentation culture is carried out at a pH value of 6.5 to 7.5 for a period of up to 50 hours; preferably, the fermentation culture is performed with agitation at a speed of 50 rpm to 150 rpm; and
(20) the method according to (17), wherein the lactic acid has a titer of at least 80 g L⁻¹, and a yield of at least 80%; preferably, the lactic acid has a chiral purity of at least 99.04%.

The present invention, compared with the prior art, has the following advantages and positive effects:

(1) The present invention proposes, for the first time, to construct a lactic acid-producing strain to increase lactic acid production through a combination of genetic manipulations, which includes: into a starting strain, 1) introducing a lactic acid synthesis pathway; 2) optimizing the lactic acid synthesis pathway; 3) inhibiting by-product synthesis pathways. Moreover, the present invention, by way of examples, demonstrates advantages after each of these manipulations. The construction strategy of the present invention is straightforward, meticulous, widely applicable and of strong utility, and the starting strain can be selected from a wide range and efficiently produce lactic acid, promoting the development of the lactic acid industry and providing a new strategy for efficiently producing lactic acid.

(2) The present invention sensibly screens for and introduces one of a D-lactate dehydrogenase gene and an L-lactate dehydrogenase gene, at the same time knocks out the other, can obtain a D- or L-lactic acid-producing strain, and significant increases the production and purity of D-lactic acid (a titer of up to 153.07 g L⁻¹, a yield of up to 93.04%, and chiral purity of D-lactic acid is up to 99.63%) and the production and purity of L-lactic acid (a titer of up to 151.12 g L⁻¹, a yield of up to 98.68%, and chiral purity of L-lactic acid is up to 99.04%, or even higher).

(3) The lactic acid-producing strain according to the present invention can be subjected to high-temperature fermentation to produce lactic acid, can leverage the advantages of high-temperature fermentation, and has high value in commercial and industrial applications.

(4) The lactic acid-producing method of the present invention needs simple culture media, is low in fermentation substrate and cultivation cost, can specifically obtain, with high productivity, D-lactic acid or L-lactic acid. The product has a single component and chiral purity of at least 99.04%, and is easy to separate and purify. It is very suitable for industrial production of lactic acid.

### Biological Material Deposit Information

The Geobacillus thermoglucosidasius strains GT6, GT8, GT9 and GT10 provided by the present invention have been deposited on Nov. 28, 2022 with the China Center for Type Culture Collection (CCTCC), deposition address: Wuhan University, Wuhan, China, zip code: 430072. The deposition number of GT6 is CCTCC M 20221822, the deposition number of GT8 is CCTCC M 20221823, the deposition number of GT9 is CCTCC M 20221824, and the deposition number of GT10 is CCTCC M 20221825.

### BRIEF DESCRIPTION OF THE DRAWING

FIG. 1 shows a schematical diagram of a method for constructing an efficient lactic acid-producing strain and use thereof according to the present invention.

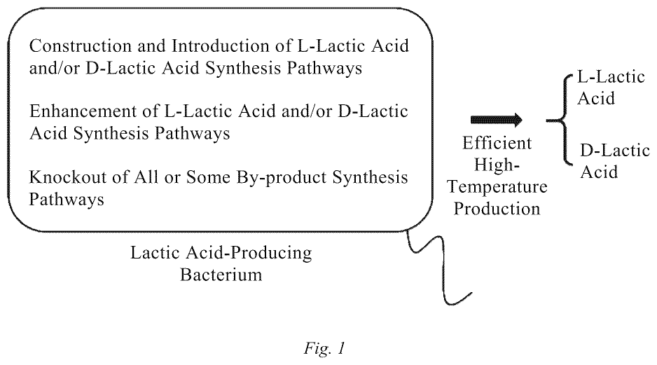

### DETAILED DESCRIPTION OF THE PREFERRED EMBODIMENTS

Below, reference is made to accompanying drawings of the specification to introduce a few preferred embodiments of the present invention so that its techniques will become more apparent and more readily understood. The present invention can be embodied in various different forms of embodiment, and the scope of protection of the present invention is not limited to the embodiments mentioned herein.

It should be understood that the technical features described above and the technical features detailed below (including, but not limited to, the embodiments and examples) can be mutually combined in an arbitrary appropriate manner, thereby creating new or preferred embodiments, as long as there is no contradiction and the combined technical solutions can be smoothly implemented and solve the problems of the present invention. The arbitrary appropriate manner is such as to be able to achieve the technical solutions of the present invention and solve the problems of the present invention and be able to achieve corresponding technical effects.

In the present invention, the terms “including”, “further”, “having”, “furthermore” and the like only mean embodiments or examples with good effects or with certain degrees of particularity. It should be understood that they do not constitute a limitation on the scope of protection of the invention.

In the present invention, “and/or” means any one or any combination of the listed items.

Numerical ranges in the present invention, unless otherwise specifically stated, all include the two endpoints.

In the present invention, “at least”, unless otherwise specifically defined, in all cases, includes the stated number.

The present invention involves temperature control, allows constant temperatures, and also allows the existence of a certain temperature variation range.

Those skilled in the art should understand that L-lactic acid of the present invention can also be referred to as L(+)-lactic acid or (S)-lactic acid, and these terms are interchangeably used; similarly, D-lactic acid can also be referred to D(−)-lactic acid or (R)-lactic acid.

In the present invention, a yield of lactic acid refers to a ratio of the produced lactic acid (the unit is gram) and the consumed carbon source (the unit is gram, for example, glucose).

In the present invention, chiral purity of lactic acid refers to the percentage of a target product (L-lactic acid or D-lactic acid) in the total of the two species of chiral lactic acid. Specifically, chiral purity of lactic acid refers to the percentage of the area of a peak for a target product (L-lactic acid or D-lactic acid) in the total area of peaks for the two species of chiral lactic acid obtained by a method for detecting chiral purity of lactic acid using a high-performance liquid chromatography system.

In the present invention, “high-temperature fermentation” is used in contrast to “medium temperature” (means lower than 37° C.). High-temperature fermentation production herein, unless otherwise specifically defined, refers to fermentation production under a 37° C. to 70° C. high temperature condition. Temperatures for high-temperature fermentation include, but are not limited to, 50° C., 55° C., 56° C., 57° C., 58° C., 59° C., 60° C., 61° C., 62° C., 63° C., 64° C., 65° C. and 70° C. Temperature ranges for high-temperature fermentation include, but are not limited to, 40° C. to 70° C., 50° C. to 65° C., 55° C. to 65° C. and 60° C. to 65° C.

As used herein, the term “starting strain” refers to a strain that has not undergone any genetic modification described in the present invention. It may be a wild-type strain, and may also be a recombinant strain that has previously experienced one or some certain known modifications.

The term “exogenous”, as used herein to refer to genes, coding sequences, proteins, enzymes and the like, refers to substances that, when in a natural state, do not belong to a starting strain. For example, an exogenous gene refers to a gene introduced from an external source into the starting strain. The exogenous gene may be a gene already in the strain's genome, and may also be a gene not in its genome. For example, according to a requirement, from an external source, into the genome of the starting strain, a gene that is already in it is introduced to overexpress the gene.

As used herein, the term “inhibit” refers to causing a function of an object being inhibited, compared to before the inhabitation is carried out, to be completely lost or weakened.

As used herein, the term “knockout” or “knockdown” refers to, by means of genetic manipulations, causing a function of a selected gene to be completely lost or weakened. The genetic manipulations include those commonly used in the art, such as insertion, substitution or deletion of one or more nucleic acid fragments from the selected gene.

The nucleotide sequences involved in the present invention are as follows:

(D-ldh coding sequence - Bacillus licheniformis):
SEQ ID No. 1
ATGAAAGTAATTTTTTTTTCTATGCACCCGTATGAAGAGGAATTTCTGGGTC
CGATTCTGCCGTCTGACTGGGACGTAGAAATGACCCCGGACTTTCTGGACG
AAACCACCGTGGAAAAGGCTAAAGGTGCCCAGGTAGTAAGCCTGTTTGTT
TCTGACAAAGCTGATGGTCCGGTACTGGAAGCGCTGCATTCTTACGGTGTG
GGCCTGCTGGCCCTGCGTTCTGCTGGCTATGATCACATCGATATTGAGACCG
CAAAACGCCTGGGTATCAAAGTAGTTAACGTGCCAGCCTATTCTCCGCACG
CTATCGCTGACCATACTCTGGCTATCATGCTGGCTCTGATTCGTCGTCTGCA
CCGTGCCCATGATAAAGTGCGCCTGGGTGATTTTGATCTGGATGGTCTGATG
GGCTTTGATCTGAACGGCAAAGTTGCTGGTGTAATTGGTCTGGGTAAAATC
GGTCGCCTGGTAGCTACCCGCCTGAAAGCGTTTGGTTGCAAAGTTCTGGGC
TATGATCCATACATTCAGCCGGAAATCGTAGAAAACGTTGATCTGGATACCC
TGATCACTCAGGCTGATATCATTTCTATTCATTGTCCGCTGACCCGTGAAAA
CTTTCATATGTTTAACGAAGAGACTTTTAAGCGTATGAAACCGGGTGCTATT
CTGGTTAACACCGCGCGTGGTGGTCTGATCGATACCAAGGCCCTGCTGGAG
GCCCTGAAGTCTGGTAAACTGGGCGGCGCAGCCCTGGATGTGTATGAATAT
GAACGTGGCCTGTTTTTTAAAAACCACCAAAAAGAAGGTATCAAAGACCC
GTATCTGGCCCAGCTGCTGGGTCTGGCCAACGTAGTGCTGACCGGTCATCA
GGCCTTTCTGACCCGTGAGGCTGTAAAAAACATCGAAGAAACTACCGTAG
AAAACATTCTGGAATGGCAAAAGAACCCGCAGGCAAAGCTGAAAAACGA
AATCTAA
(D-ldh^{P101Q} coding sequence - Bacillus licheniformis):
SEQ ID No. 2
ATGAAAGTAATTTTTTTTTCTATGCACCCGTATGAAGAGGAATTTCTGGGTC
CGATTCTGCCGTCTGACTGGGACGTAGAAATGACCCCGGACTTTCTGGACG
AAACCACCGTGGAAAAGGCTAAAGGTGCCCAGGTAGTAAGCCTGTTTGTT
TCTGACAAAGCTGATGGTCCGGTACTGGAAGCGCTGCATTCTTACGGTGTG
GGCCTGCTGGCCCTGCGTTCTGCTGGCTATGATCACATCGATATTGAGACCG
CAAAACGCCTGGGTATCAAAGTAGTTAACGTGCCAGCCTATTCTCAGCACG
CTATCGCTGACCATACTCTGGCTATCATGCTGGCTCTGATTCGTCGTCTGCA
CCGTGCCCATGATAAAGTGCGCCTGGGTGATTTTGATCTGGATGGTCTGATG
GGCTTTGATCTGAACGGCAAAGTTGCTGGTGTAATTGGTCTGGGTAAAATC
GGTCGCCTGGTAGCTACCCGCCTGAAAGCGTTTGGTTGCAAAGTTCTGGGC
TATGATCCATACATTCAGCCGGAAATCGTAGAAAACGTTGATCTGGATACCC
TGATCACTCAGGCTGATATCATTTCTATTCATTGTCCGCTGACCCGTGAAAA
CTTTCATATGTTTAACGAAGAGACTTTTAAGCGTATGAAACCGGGTGCTATT
CTGGTTAACACCGCGCGTGGTGGTCTGATCGATACCAAGGCCCTGCTGGAG
GCCCTGAAGTCTGGTAAACTGGGCGGCGCAGCCCTGGATGTGTATGAATAT
GAACGTGGCCTGTTTTTTAAAAACCACCAAAAAGAAGGTATCAAAGACCC
GTATCTGGCCCAGCTGCTGGGTCTGGCCAACGTAGTGCTGACCGGTCATCA
GGCCTTTCTGACCCGTGAGGCTGTAAAAAACATCGAAGAAACTACCGTAG
AAAACATTCTGGAATGGCAAAAGAACCCGCAGGCAAAGCTGAAAAACGA
AATCTAA
(codon-optimized sequence of SEQ ID No. 2 for
Geobacillus thermoglucosidasius):
SEQ ID No. 3
ATGAAAGTAATTTTTTTTAGCATGCATCCGTATGAAGAAGAATTTTTAGGCC
CGATTTTACCGTCGGATTGGGATGTAGAAATGACGCCGGATTTTTTAGATGA
AACGACGGTGGAAAAAGCGAAAGGAGCGCAAGTAGTAAGCTTGTTTGTTT
CGGATAAAGCGGATGGCCCGGTACTTGAAGCGCTTCATTCGTATGGAGTGG
GCCTTTTGGCGCTTCGCAGCGCGGGCTATGATCATATTGATATTGAAACAGC
GAAACGCCTGGGCATTAAAGTAGTTAATGTGCCGGCGTATTCGCAACATGC
GATTGCGGATCATACATTAGCGATTATGCTTGCGCTTATTCGCCGCCTTCATC
GCGCGCATGATAAAGTGCGCCTGGGAGATTTTGATCTTGATGGCCTTATGGG
CTTTGATTTAAATGGCAAAGTTGCGGGCGTAATTGGCCTTGGCAAAATTGG
CCGCCTGGTAGCGACACGCTTAAAAGCGTTTGGCTGCAAAGTTTTAGGCTA
TGATCCGTATATTCAACCGGAAATTGTAGAAAATGTTGATTTGGATACACTTA
TCACACAAGCGGATATCATTTCGATTCATTGTCCGCTTACGCGCGAAAATTT
TCATATGTTTAATGAAGAAACATTTAAACGCATGAAACCGGGCGCGATTTTG
GTTAACACGGCGCGCGGAGGCCTTATAGATACAAAAGCGTTGCTTGAAGCG
TTAAAATCGGGAAAACTTGGCGGCGCGGCGCTTGATGTGTATGAATATGAA
CGCGGCCTTTTTTTTAAAAACCATCAAAAAGAAGGCATTAAAGATCCGTAT
CTTGCGCAACTTTTGGGCTTGGCGAATGTAGTGTTAACAGGCCATCAAGCG
TTTCTTACGCGCGAAGCGGTAAAAAACATTGAAGAAACAACAGTAGAAAA
TATTTTAGAATGGCAAAAAAATCCGCAAGCGAAACTTAAAAATGAAATCTG
A
(D-ldhP^{101N} coding sequence - Bacillus licheniformis):
SEQ ID No. 4
ATGAAAGTAATTTTTTTTTCTATGCACCCGTATGAAGAGGAATTTCTGGGTC
CGATTCTGCCGTCTGACTGGGACGTAGAAATGACCCCGGACTTTCTGGACG
AAACCACCGTGGAAAAGGCTAAAGGTGCCCAGGTAGTAAGCCTGTTTGTT
TCTGACAAAGCTGATGGTCCGGTACTGGAAGCGCTGCATTCTTACGGTGTG
GGCCTGCTGGCCCTGCGTTCTGCTGGCTATGATCACATCGATATTGAGACCG
CAAAACGCCTGGGTATCAAAGTAGTTAACGTGCCAGCCTATTCTAACCACG
CTATCGCTGACCATACTCTGGCTATCATGCTGGCTCTGATTCGTCGTCTGCA
CCGTGCCCATGATAAAGTGCGCCTGGGTGATTTTGATCTGGATGGTCTGATG
GGCTTTGATCTGAACGGCAAAGTTGCTGGTGTAATTGGTCTGGGTAAAATC
GGTCGCCTGGTAGCTACCCGCCTGAAAGCGTTTGGTTGCAAAGTTCTGGGC
TATGATCCATACATTCAGCCGGAAATCGTAGAAAACGTTGATCTGGATACCC
TGATCACTCAGGCTGATATCATTTCTATTCATTGTCCGCTGACCCGTGAAAA
CTTTCATATGTTTAACGAAGAGACTTTTAAGCGTATGAAACCGGGTGCTATT
CTGGTTAACACCGCGCGTGGTGGTCTGATCGATACCAAGGCCCTGCTGGAG
GCCCTGAAGTCTGGTAAACTGGGCGGCGCAGCCCTGGATGTGTATGAATAT
GAACGTGGCCTGTTTTTTAAAAACCACCAAAAAGAAGGTATCAAAGACCC
GTATCTGGCCCAGCTGCTGGGTCTGGCCAACGTAGTGCTGACCGGTCATCA
GGCCTTTCTGACCCGTGAGGCTGTAAAAAACATCGAAGAAACTACCGTAG
AAAACATTCTGGAATGGCAAAAGAACCCGCAGGCAAAGCTGAAAAACGA
AATCTAA
(L-ldh coding sequence - Bacillus coagulans H-2 strain):
SEQ ID No.5
ATGAAAAAAGTAAACCGTGTTGCAGTGATCGGAACTGGCGCAGTAGGCAC
AAGTTATTGCTATGCAATGATTAACCAGGGTGTTGCAGAAGAGCTTGTTTTA
ATCGATATTAACGAAGCAAAAGCAGAAGGGGAAGCCATGGACCTGAACCA
CGGCCTGCCATTTGCGCCTACGCCGACCCGCGTTTGGAAAGGCGATTATTC
CGATTGCGGCACTGCCGACCTTGTTGTCATTACGGCAGGTTCCCCGCAAAA
ACCGGGCGAAACAAGGCTTGATCTTGTTTCCAAAAACGCAAAAATTTTTAA
AGGCATGATTAAGAGCATCATGGACAGCGGCTTTAACGGGATTTTTCTTGTT
GCCAGCAACCCGGTTGACATTTTGACATATGTAACTTGGAAAGAGTCCGGC
CTGCCGAAAGAACATGTTATCGGTTCGGGCACAGTGCTTGACTCCGCGCGT
CTCCGCAACTCTTTGAGCGCCCAATTTGGAATTGACCCGCGCAATGTGCAT
GCTGCGATTATCGGCGAACACGGCGATACGGAACTTCCGGTATGGAGCCAT
ACAACTATCGGTTACGATACGATTGAAAGTTATCTACAAAAAGGAATTATTG
ACGAAAAGACGTTAGATGACATTTTTGTCAATACGAGAGATGCGGCTTATC
ATATTATTGAACGAAAAGGGGCCACATTTTACGGCATCGGGATGTCCCTGAC
CCGGATTACAAGGGCAATCCTGAACAATGAAAACAGCGTATTGACGGTCTC
TGCATTTCTTGAAGGCCAATACGGAAACAGCGATGTGTACGTTGGCGTTCC
GGCCATCATCAATCGCCAGGGCATCCGTGAAGTGGTTGAAATCAAACTGAA
CGAAAAAGAACAGGAACAGTTCAATCATTCTGTAAAAGTGCTAAAAGAAA
CAATGGCACCGATATTGTAA
(pfk and pyk coding sequence - Bacillus coagulans H-2 strain)
SEQ ID No. 6
ATGAAGCGAATTGGAGTATTGACAAGCGGCGGCGATGCACCGGGGATGAA
TGCGGCGGTCCGCGCGATTGCCCGTAAAGGGATTTATCACGGCCTGGAAGT
TTACGGCATTCGCCAAGGTTATAACGGATTGATTCAAGGAAACATCCAAAA
GCTCGAAGCAGGATCTGTTGGCGATATTCTCCAGCGGGGCGGCACGGTTTT
GCAGTCGGCAAGAAGCGAAGAATTCAAAACGCCGGAAGGGCAGCAAAAA
GCGATCAGGCAGCTGAAAGACCATGGCATTGAAGCGCTCGTTGTGATCGG
CGGCGACGGTTCCTACCAAGGGGCCAAAAAGTTGACGGAACAGGGCTTTA
ACTGCATTGGTGTGCCAGGGACAATCGATAACGACATCCCGGGGACGGATT
TTACAATCGGTTTTGATACGGCATTGAACACAGTGCTTGATGCGATTGATAA
AATTCGCGACACCGCTTCTTCCCACGAACGCACCTTTATTATTGAAGTCATG
GGCAGAAATGCCGGGGATATCGCGCTCTGGTCCGGCCTGGCCGGCGGAGC
CGAATCGATTATTATTCCGGAAGAAAAATATGACTTAAAAGATGTCGTGGAG
CGTCTTGAACAGGGGAGAAAACGCGGCAAACGCCACAGCATCATCATTGT
CGCGGAAGGCGTGATGAGCGGCAACGAGTTTGCTGAACAATTGAAAAAAA
CCGGTGTGATCGGCGATACCCGCGTTTCTGTTCTCGGCCATATCCAGCGCGG
CGGTTCTCCGACGGCATTTGACCGCGTGCTTGCAAGCCGCCTCGGCGCAA
GGGCTGTTGAACTGCTGCTTGAAGGAAAAGGGGGCCGCGCTGTCGGCATT
CAAAATAACCAGCTGGTTGACCACGATATCCTTGAGATTCTCGGAAAACCG
CACGCCGTTAATAAAAACATGTACAAGCTGTCGAAAGAATTGTCGATCTAA
CGTTTCTTAGGAGGAAATAAAATGAAAAAAACCAAAATTGTATGTACAATC
GGACCTGCCAGTGAAAGTGTGGAAATGCTTGAAAGATTAATGGCAAACGG
GATGGATGTTTGCCGCCTGAACTTCTCGCACGGCAGCCATGAGGAACATCT
TGCCCGGATTAAAAATATCCGTGAAGCTGCAAAAAACCAAAACAAAACGA
TCGGGCTTCTGCTCGATACAAAGGGCCCGGAAATCCGCACCCATGATATGA
AAGACGGCGGATTCGAGCTCGTTGAAGGCATGACACCGGTCATTTCAATGA
CAGAAGTGCTCGGGACACCGGAAAAATTTTCGGTCACATATGAAGGGCTG
ATTGATGATGTGCACGTTGGCTCTAAAATTTTACTTGATGACGGTTTGATTG
AACTGGAAGTGACGGCCATCGATAAAAACGCCGGTGAAATCCATACAAAA
GTGCTGAACCGCGGCGTTTTGAAAAACAAAAAAGGTGTTAACGTCCCGGG
TGTTTCCGTGAACCTTCCGGGCATCACCGAAAAAGACGTGAGCGATATCCT
GTTCGGGCTTGAACAAGGCATTGACTTCATTGCGGCTTCGTTTGTACGCCG
GCCGTCCGACGTTTTGGAAATCCGCCAGCTCCTTGAAGAACACGATGCTTT
GCATGTGAAAATTTTCCCTAAAATTGAAAACCAGGAAGGCGTCGACAATAT
CGATGAAATCCTTGCGGTATCAGACGGCTTAATGGTTGCCCGCGGCGACCT
CGGCGTTGAAATTCCGACCGAAGCGGTGCCGCTCGTACAAAAAGAAATGA
TCAGAAAATGTAATACGCTCGGCAAACCGGTGATTACCGCAACGCAAATGC
TTGATTCGATGCAACGCAACCCGCGCCCGACCCGCGCGGAAGCAAGCGAC
GTGGCCAACGCCATTTTTGACGGCACGGATGCGATCATGCTTTCCGGCGAA
ACGGCAGCCGGGAAATATCCTGCTGAAGCGGTTAAGACGATGTACAATATT
GCGGTTCATGTGGAAAAAGCAATTAACCATCGCGATATTCTGAACAAGCGC
AGCAAGAGCACGGACCATAATATGACAGACGCTATCTGCCAGTCCGTTGCC
CATACGGCTTTAAATCTTGATGTGAATGCCATTATTGCGCCGACTGAAAGCG
GCTATACGGCACGCATGATCTCCAAATACCGCCCGGCGGCCCCAATCATTGC
TGTCACGAGCGATCCGAAAGTACAACGCGGCTTAACTGTTGTGTCCGGCGT
ATACCCACAATTGGGCACAAAGGCAAACAATACGGATGAAATGCTTGAAAT
TGCAGTGGAGGAAGCGTTGAAATCCGAAATCGTCCATCACGGCGACCTTG
TGATCATTACAGCAGGCGTCCCGGTTGGTGGGAAAGGCACCACCAACCTG
ATGAAAGTGCACCTGATCGGTGATATATTGGCAAAAGGCCAGGGAATCGGC
AGAAAATCGGCATTCGGCCCGGTCATCGTTGCTGAAAGCCCTGAAGAAGC
AAACGCAAAGGCAACAGAAGGTTGTGTGCTCGTCACGAGAACGACCGAC
AAAGAAATCATGCCGGCCATTGAAAAATGCGCCGCGCTGATTACGGAAGA
AGGCGGCTTGACAAGCCATGCTGCAGTTGTTGGCATCAATGTCGGCATTCC
GGTCATTGTTGGCGTTGAAAAAGCCGTTTCCATTTTTGAAGACGGGCAGGA
AGTTACGGTAGATGCGGCAACCGGCTCGGTTTACAACGGCCATGCGACTGT
ATTGTAA
(P_{ldh} promoter - Geobacillus stearothermophilus):
SEQ ID No. 7
GCGGCCGCACTAGTGCGGGACGGGGAGCTGAGTGCTCCCGTTGTTTGCCG
CGGCGTCTGTCATGAAATGGACAAACAATAGTCAAACAATCGCCACAATCG
CGCATGCATTGCGGTGCGCCTTTCGCGTAAAATATTTATATGAAAGTGTTCG
CATTATATTGAAGGAGGATGAATGCA
(L-ldh coding sequence - Geobacillus thermoglucosidasius
DSM 2542)
SEQ ID No. 8
ATGAAACAACAAGGCATGAATCGAGTAGCACTTATAGGAACGGGGTTCGTT
GGGGCCAGCTATGCATTTGCCCTTATGAACCAAGGAATAGCAGATGAGTTA
GTATTGATTGATGTAAATAAGAATAAGGCAGAGGGCGATGTGATGGATTTAA
ATCACGGAAAAGTATTCGCGCCGAAGCCGATGAATATTTGGTTTGGAGATTA
TCAAGATTGCCAAGACGCCGATTTGGTGGTGATTTGTGCAGGGGCTAACCA
AAAGCCGGGAGAAACAAGACTGGATCTTGTTGACAAAAATATTAATATCTT
CAAAACGATTGTCGATTCTGTGATGAAATCCGGATTTGATGGCGTTTTTCTT
GTGGCAACGAACCCAGTGGATATTTTAACGTATGCTACTTGGAAATTTAGCG
GGTTACCGAAAGAGCGGGTAATCGGCTCAGGAACGATTCTTGATACAGCAA
GATTCCGCTTCTTGCTAAGTGAATATTTTCAAGTGGCTCCGACCAATGTACA
TGCGTATATTATTGGCGAGCATGGGGATACAGAGCTGCCTGTTTGGAGCCAT
GCGGAAATTGGAAGCATTCCAGTTGAGCAAATATTGATGCAAAACGATAAC
TATAGAAAAGAGGATTTAGACAATATCTTTGTTAATGTTCGTGATGCGGCAT
ATCAAATCATTGAGAAAAAAGGGGCAACGTATTACGGCATTGCAATGGGAT
TAGTCCGTATCACTCGTGCTATTTTGCACAATGAAAATGCCATCTTAACCGT
TTCTGCTCATTTGGACGGCCAATATGGCGAACGAAATGTTTATATTGGCGTG
CCTGCCATTATCAACCGAAACGGTATTCGTGAAGTGATGGAATTGACGCTA
AATGAAACAGAACAACAACAATTCCATCATAGTGTAACTGTATTAAAAGAC
ATTCTTTCCCGTTATTTTGATGATGTAAAATAA

The amino acid sequences involved in the present invention are as follows.

(D-ldh^{P101Q} amino acid sequence):
SEQ ID No. 9
MKVIFFSMHPYEEEFLGPILPSDWDVEMTPDFLDETTVEKAKGAQVVSLF
VSDKADGPVLEALHSYGVGLLALRSAGYDHIDIETAKRLGIKVVNVPAYS
QHAIADHTLAIMLALIRRLHRAHDKVRLGDFDLDGLMGFDLNGKVAGVIG
LGKIGRLVATRLKAFGCKVLGYDPYIQPEIVENVDLDTLITQADIISIHC
PLTRENFHMFNEETFKRMKPGAILVNTARGGLIDTKALLEALKSGKLGGA
ALDVYEYERGLFFKNHQKEGIKDPYLAQLLGLANVVLTGHQAFLTREAVK
NIEETTVENILEWQKNPQAKLKNEI
(D-ldh^{P101N} amino acid sequence):
SEQ ID No. 10
MKVIFFSMHPYEEEFLGPILPSDWDVEMTPDFLDETTVEKAKGAQVVSLF
VSDKADGPVLEALHSYGVGLLALRSAGYDHIDIETAKRLGIKVVNVPAYS
NHAIADHTLAIMLALIRRLHRAHDKVRLGDFDLDGLMGFDLNGKVAGVIG
LGKIGRLVATRLKAFGCKVLGYDPYIQPEIVENVDLDTLITQADIISIHC
PLTRENFHMFNEETFKRMKPGAILVNTARGGLIDTKALLEALKSGKLGGA
ALDVYEYERGLFFKNHQKEGIKDPYLAQLLGLANVVLTGHQAFLTREAVK
NIEETTVENILEWQKNPQAKLKNEI

The sequence shown in SEQ ID No. 1 is the sequence of the D-lactate dehydrogenase gene of Bacillus licheniformis.

The sequence shown in SEQ ID No. 2 is an encoding sequence of D-ldh^{P101Q}. It results from mutation of the codon in the nucleotide sequence of SEQ ID No. 1 that encodes the amino acid at position 101 into a glutamine-coding codon.

The sequence shown in SEQ ID No. 3 is a sequence from codon-optimization of the coding sequence as shown in SEQ ID No. 2 for Geobacillus thermoglucosidasius as a host.

The sequence shown in SEQ ID No. 4 is an encoding sequence of D-ldh^{P101N} It results from mutation of the codon in the nucleotide sequence of SEQ ID No. 1 that encodes the amino acid at position 101 into an asparagine-coding codon.

The sequence shown in SEQ ID No. 5 is an encoding sequence of L-lactate dehydrogenase in a Bacillus coagulans H-2 strain.

The sequence shown in SEQ ID No. 6 is an encoding sequence of 6-phosphofructokinase and pyruvate kinase in a Bacillus coagulans H-2 strain.

The sequence shown in SEQ ID No. 7 is a promoter sequence upstream of the L-lactate dehydrogenase gene of Geobacillus stearothermophilus.

The sequence shown in SEQ ID No. 8 is an encoding sequence of L-lactate dehydrogenase in Geobacillus thermoglucosidasius DSM 2542.

The sequence shown in SEQ ID No. 9 is the amino acid sequence of D-ldh^{P101Q}.

The sequence shown in SEQ ID No. 10 is the amino acid sequence of D-ldh^{P101N}.

Information of sequences of primers used in the present invention is as shown in Table 1 below.

TABLE 1
Information of Sequences of Primers Used in Present Invention

| Primer | Sequence (5′-3′) |
|---|---|
| pUBTY-F | AGGAATATTCAGCAATTTGCCC |
| (SEQ ID No. 11) | |
| pUBTY-R | CAGCTGGCACGACAGGTTT |
| (SEQ ID No. 12) | |
| LDH-Up-F | tagttagttagcccttagtgactcgGCTTGCATCTTTCGCTGCA |
| (SEQ ID No. 13) | |
| LDH-Up-R | AAAAATTACTTTCATCGCTGTCTGTCATCCTTTCCA |
| (SEQ ID No. 14) | |
| LDH-DLDH-F | GGATGACAGACAGCGATGAAAGTAATTTTTTTTTCTATGCAC |
| (SEQ ID No. 15) | |
| LDH-DLDH-R | TATTCAAAGTCAGTATTAGATTTCGTTTTTCAGCTTTGC |
| (SEQ ID No. 16) | |
| LDH-Down-F | AAAAACGAAATCTAATACTGACTTTGAATACAACAAGGTGA |
| (SEQ ID No. 17) | |
| LDH-Down-R | aacagctatgaccatgattacgccaTTAATACCCTTCCACTTATCCAAGA |
| (SEQ ID No. 18) | |
| LDH-YZ-F | TCCGGTGCACAGCTTGTTTC |
| (SEQ ID No. 19) | |
| LDH-YZ-R | AGCTGTATTTGCATTTTTCTCCG |
| (SEQ ID No. 20) | |
| GPFYK-Up-F | tagttagttagcccttagtgactcgGCAACGCGTATGCTGAATAGTAA |
| (SEQ ID No. 21) | |
| GLFYK-Up-R | CACTAGTGCGGCCGCCCGCTATCAGCTTCTTAATGTTGT |
| (SEQ ID No. 22) | |
| GLFYK-Pl-F | AGAAGCTGATAGCGGGCGGCCGCACTAGTGCG |
| (SEQ ID No. 23) | |
| GLFYK-Pl-R | TCCAATTCGCTTCATTGCATTCATCCTCCTTCAATATAAT |
| (SEQ ID No. 24) | |
| GLFYK-F | AGGAGGATGAATGCAATGAAGCGAATTGGAGTATTGACA |
| (SEQ ID No. 25) | |
| GPFYAK-R | CTGATAGCAACTTCCTTACAATACAGTCGCATGGCC |
| (SEQ ID No. 26) | |
| GPFYAK-DOWN-F | GCGACTGTATTGTAAGGAAGTTGCTATCAGCTTTTTCTTT |
| (SEQ ID No. 27) | |
| GPFYK-DOWN-R | aacagctatgaccatgattacgccaGATTCATCTCAGCGGAAACTACTAA |
| (SEQ ID No. 28) | |
| GFYK-YZ-F | CGATCGACGTTTGTGATTGACC |
| (SEQ ID No. 29) | |
| GFYK-YZ-R | TATCCAATCAATCGCTTTCCG |
| (SEQ ID No. 30) | |
| AcDH1-Up-F | tagttagttagcccttagtgactcgCACTTCTGTTGGAGCGTTTCC |
| (SEQ ID No. 31) | |
| AcDH1-Up-R | AAAGGGGACAATTTCATGAAATTCCCAAGGTTAACAGATA |
| (SEQ ID No. 32) | |
| AcDH1-Down-F | CCTTGGGAATTTCATGAAATTGTCCCCTTTCAGTCTGA |
| (SEQ ID No. 33) | |
| AcDH1-Down-R | aacagctatgaccatgattacgccaGGCGCCTCGACCATGTTAA |
| (SEQ ID No. 34) | |
| AcDH1-YZ-F | AAGAAAACTATTATACACACCCGCA |
| (SEQ ID No. 35) | |
| AcDH1-YZ-R | CATGGTGAGGCCTATCAGTTTAC |
| (SEQ ID No. 36) | |
| AcDH2-Up-F | tagttagttagcccttagtgactcgGCGCAGACTGCCGTCAAT |
| (SEQ ID No. 37) | |
| AcDH2-Up-R | GGAAGGGGAATGGACTCGAAACGAAAGGAGCGG |
| (SEQ ID No. 38) | |
| AcDH2-Down-F | CTCCTTTCGTTTCGAGTCCATTCCCCTTCCCATATAA |
| (SEQ ID No. 39) | |
| AcDH2-Down-R | aacagctatgaccatgattacgccaCCATCGTTGGCAAAAAAATTG |
| (SEQ ID No. 40) | |
| AcDH2-YZ-F | CTTAACACCTGTTTCATATCCAAGG |
| (SEQ ID No. 41) | |
| AcDH2-YZ-R | GAACATTACAGAAATCGCCGC |
| (SEQ ID No. 42) | |
| AADH-Up-F | tagttagttagcccttagtgactcgCAAGCTATATGCTCAAGACATCGA |
| (SEQ ID No. 43) | |
| AADH-Up-R | GAGTGGTTTTTATTTACGCATTCTCCCTCCTGATTG |
| (SEQ ID No. 44) | |
| AADH-Down-F | GGAGGGAGAATGCGTAAATAAAAACCACTCCCCCAAA |
| (SEQ ID No. 45) | |
| AADH-Down-R | aacagctatgaccatgattacgccaCGTTTATCAACGCTTCCAAT |
| (SEQ ID No. 46) | |
| AADH-YZ-F | CGACAAAACAGATTATCGCCC |
| (SEQ ID No. 47) | |
| AADH-YZ-R | AACGCCGATACGTGGGAAA |
| (SEQ ID No. 48) | |
| DLDH-Up-R | AAAAATTACTTTCATACGCATTCTCCCTCCTGATTG |
| (SEQ ID No. 49) | |
| DLDH-F | GGAGGGAGAATGCGTATGAAAGTAATTTTTTTTAGCATGCA |
| (SEQ ID No. 50) | |
| DLDH-R | GAGTGGTTTTTATTTTCAGATTTCATTTTTAAGTTTCGCT |
| (SEQ ID No. 51) | |
| DLDH-Down-F | AAAAATGAAATCTGAAAATAAAAACCACTCCCCCAAA |
| (SEQ ID No. 52) | |
| pflB-Up-F | tagttagttagcccttagtgactcgAATTCCAGGCGTCGCCC |
| (SEQ ID No. 53) | |
| pflB-Up-R | AAAAATTACTTTCATAACAGTTTCCCTCCCATGCAT |
| (SEQ ID No. 54) | |
| DLDH-F1 | GGGAGGGAAACTGTTATGAAAGTAATTTTTTTTAGCATGCA |
| (SEQ ID No. 55) | |
| DLDH-R1 | GGAGGGGGGAGATTATCAGATTTCATTTTTAAGTTTCGCT |
| (SEQ ID No. 56) | |
| pflB-Down-F | AAAAATGAAATCTGATAATCTCCCCCCTCCTTCTTAAG |
| (SEQ ID No. 57) | |
| pflB-Down-R | aacagctatgaccatgattacgccaCATTCTTTAATGTGCGAATAAAAGC |
| (SEQ ID No. 58) | |
| pflB-YZ-F | TTAACGGCCGCTTTGTCTTC |
| (SEQ ID No. 59) | |
| pflB-YZ-R | GCTTTCTTCCGAAGGAGGCTC |
| (SEQ ID No. 60) | |
| pta-Up-F | tagttagttagcccttagtgactcgTAGAAGCAAACGTGTTTCGTTTT |
| (SEQ ID No. 61) | |
| pta-Up-RI | TCACCTTTTTCTGAAGAACGAATCCTCCCTAATGTTTG |
| (SEQ ID No. 62) | |
| pta-Down-F1 | AGGGAGGATTCGTTCTTCAGAAAAAGGTGACGAAACG |
| (SEQ ID No. 63) | |
| pta-Down-R | aacagctatgaccatgattacgccaAGCCGCTGCCGTTTATACATA |
| (SEQ ID No. 64) | |
| pta-YZ-F | GCATATTTCCGTCCTGTCATGC |
| (SEQ ID No. 65) | |
| pta-YZ-R | CTCGGAACAATCGTCGGGTAT |
| (SEQ ID No. 66) | |
| LLDH-Up-R1 | GTTTACTTTTTTCATACGCATTCTCCCTCCTGATTG |
| (SEQ ID No. 67) | |
| LLDH-F1 | GGAGGGAGAATGCGTATGAAAAAAGTAAACCGTGTTGCA |
| (SEQ ID No. 68) | |
| LLDH-R1 | GAGTGGTTTTTATTTTTACAATATCGGTGCCATTGTTTC |
| (SEQ ID No. 69) | |
| LLDH-Down-F1 | GCACCGATATTGTAAAAATAAAAACCACTCCCCCAAA |
| (SEQ ID No. 70) | |

The present invention has found that lactic acid-producing strains constructed by combinations of genetic modifications proposed in the present invention can maximumize lactic acid production. When a high-temperature fermentation strain is used as a starting strain, the advantages of high-temperature fermentation can be further combined.

On the basis of this concept, in a first aspect of the present invention, there is provided a method for constructing a lactic acid-producing strain, characterized by genetically engineering a starting strain to increase lactic acid production, which comprises: 1) introducing a lactic acid synthesis pathway; 2) optimizing the lactic acid synthesis pathway; 3) inhibiting by-product synthesis pathways.

Wherein, the lactic acid is L-lactic acid or D-lactic acid.

In some embodiments of the present invention, the starting strain is a lactic acid-and/or pyruvic acid-producing strain; preferably, the starting strain shows growth activity under a 37° C. to 70° C. temperature condition, can withstand a temperature of 37° C. to 70° C., and can produce lactic acid and/or pyruvic acid at a temperature of 37° C. to 70° C.

In some embodiments of the present invention, the starting strain includes microorganisms of genus Bacillus and Geobacillus. Preferably, the starting strain includes Bacillus lichenformis, Bacillus coagulans, Bacillus smithii, Bacillus pallidus and Geobacillus thermoglucosidasius.

In some specific preferred embodiments, the starting strain is Geobacillus thermoglucosidasius.

In the method of the present invention for constructing a lactic acid-producing strain, genetically engineering the starting strain comprises introducing a lactic acid synthesis pathway. In some embodiments, introducing the lactic acid synthesis pathway comprises introducing, into the starting strain, an L-lactate dehydrogenase gene L-ldh or a D-lactate dehydrogenase gene D-ldh.

In the method of the present invention for constructing a lactic acid-producing strain, genetically engineering the starting strain further comprises optimizing the lactic acid synthesis pathway. In some embodiments, optimizing the lactic acid synthesis pathway comprises introducing, into the starting strain, coding sequences of one or more genes selected from: a lactate dehydrogenase gene ldh, a 6-phosphofructokinase gene pfk and a pyruvate kinase gene pyk, wherein the lactate dehydrogenase gene ldh is an L-lactate dehydrogenase gene L-ldh or a D-lactate dehydrogenase gene D-ldh.

In some embodiments of the present invention, the L-lactate dehydrogenase gene L-ldh is derived from Bacillus coagulans and/or Geobacillus thermoglucosidasius, the D-lactate dehydrogenase gene D-ldh is derived from Bacillus lichenformis, the 6-phosphofructokinase gene pfk is derived from Bacillus coagulans and the pyruvate kinase gene pyk is derived from Bacillus coagulans.

According to the present invention, introducing the coding sequences of the target genes into the starting strain comprises integrating the coding sequences of the target genes into a genome of the starting strain, or expressing them in the form of a plasmid in the starting strain. Depending on required expression strength of each inserted gene, single-copy or multi-copy of its coding sequence may be introduced into the starting strain.

If the coding sequences of multiple target genes are introduced, the introduced coding sequences of the multiple target genes may be introduced into the starting strain as individual single-gene expression fragments, and may be introduced into the starting strain as tandem expression fragments.

In the method of the present invention for constructing a lactic acid-producing strain, genetically engineering the starting strain further comprises inhibiting by-product synthesis pathways. In some embodiments of the method for constructing an L-lactic acid-producing strain, the by-products include D-lactic acid, formic acid, acetic acid and ethanol. In some embodiments of the method for constructing a D-lactic acid-producing strain, the by-products include L-lactic acid, formic acid, acetic acid and ethanol.

In some embodiments, inhibiting the by-product synthesis pathways comprises knocking out or down one or more coding genes in the starting strain selected from: a lactate dehydrogenase gene ldh, a formate acetyltransferase gene pflB, an acetaldehyde dehydrogenase gene acdh, an aldehyde-alcohol dehydrogenase gene aadh and a phosphate acetyltransferase gene pta, wherein the lactate dehydrogenase gene ldh is an L-lactate dehydrogenase gene L-ldh or a D-lactate dehydrogenase gene D-ldh.

The genomes of some of the microorganisms contain do not contain only one acetaldehyde dehydrogenase gene acdh. In the present invention, one or more may be knocked out or knocked down, preferably all are knocked out or knocked down, of the acetaldehyde dehydrogenase genes acdh. Different acetaldehyde dehydrogenase genes acdh may be distinguished by acdh1, acdh2, etc.

According to the genetic engineering strategy of the present invention, if it is desired to obtain a D-lactic acid-producing strain, then into the starting strain, a D-lactic acid synthesis pathway is introduced and optimized, and an L-lactic acid synthesis pathway is inhibited; and vice versa.

For example, in some embodiments of the present invention, into the starting strain, a D-lactate dehydrogenase gene D-ldh, a 6-phosphofructokinase gene pfk and a pyruvate kinase gene pyk are introduced, an L-lactate dehydrogenase gene L-ldh, an acetaldehyde dehydrogenase gene acdh and an aldehyde-alcohol dehydrogenase gene aadh of the starting strain are knocked out or knocked down, and preferably, a formate acetyltransferase gene pflB and a phosphate acetyltransferase gene pta are knocked out or knocked down, thereby constructing a D-lactic acid-producing strain.

In some other embodiments of the present invention, into the starting strain, an L-lactate dehydrogenase gene L-ldh, a 6-phosphofructokinase gene pfk and a pyruvate kinase gene pyk are introduced, a D-lactate dehydrogenase gene D-ldh, an acetaldehyde dehydrogenase gene acdh and an aldehyde-alcohol dehydrogenase gene aadh of the starting strain are knocked out or knocked down, and preferably, a formate acetyltransferase gene pflB and a phosphate acetyltransferase gene pta are knocked out or knocked down, thereby constructing an L-lactic acid-producing strain.

The invention has further found that mutating position 101 of D-lactate dehydrogenase into glutamine or asparagine can significantly improve catalytic performance of D-lactate dehydrogenase and that introducing it into the starting strain can further significantly increase lactic acid production. Accordingly, in a preferred embodiment of the present invention, into the starting strain, a mutant D-lactate dehydrogenase gene is introduced. It may be introduced in the step of introducing the lactic acid synthesis pathway, may also be introduced in the step of optimizing the lactic acid synthesis pathway, and may also be introduced in both steps. The mutant D-lactate dehydrogenase gene encodes the amino acid sequence as shown in SEQ ID No. 9 or SEQ ID No. 10, or encodes an amino acid sequence exhibiting 30% or higher identity to the amino acid sequence shown in SEQ ID No. 9 or SEQ ID No. 10.

In some specific preferred embodiments, the method of the present invention for constructing a D-lactic acid-producing strain includes sequence-independent steps of:
1) selecting a starting strain, such as Geobacillus thermoglucosidasius, and using the polymerase chain reaction (PCR) technique and genetic manipulation methods to knock out an L-ldh gene and introduce a D-ldh gene at the same position;
2) overexpressing a pfk gene and a pyk gene;
3) knocking out an acdh gene;
4) knocking out an aadh gene; and
5) inserting a codon-optimized D-ldh gene (e.g., the gene as shown in SEQ ID No. 3).

In other preferred embodiments, the method of the present invention for constructing a D-lactic acid-producing strain further includes steps of:
6) knocking out a pflB gene and inserting a codon-optimized D-ldh gene (e.g., the gene as shown in SEQ ID No. 3); and
7) knocking out a pta gene.

In some specific preferred embodiments, the method of the present invention for constructing an L-lactic acid-producing strain includes sequence-independent steps of:
1) selecting a starting strain, such as Geobacillus thermoglucosidasius, and using the polymerase chain reaction (PCR) technique and genetic manipulation methods to knock out a D-ldh gene and introduce an L-ldh gene at the same position;
2) overexpressing a pfk gene and a pyk gene;
3) knocking out an acdh gene;
4) knocking out an aadh gene;
5) inserting an L-ldh gene; and
6) preferably, knocking out a pflB gene and a pta gene.

In some specific preferred embodiments, the method of the present invention for constructing an L-lactic acid-producing strain may also, directly with a constructed efficient D-lactic acid-producing strain as a basis, knock out a D-ldh gene and introduce an L-ldh gene, thereby easily constructing an efficient L-lactic acid-producing strain.

In a second aspect of the present invention, there is provided a lactic acid-producing strain characterized by being constructed according to a method of the present invention as described in the present invention.

In some embodiments of the present invention, the lactic acid-producing strain is Geobacillus thermoglucosidasius.

In a preferred embodiment of the present invention, the lactic acid-producing strain is selected from D-lactic acid-producing strains GT6, GT7 and GT8 and L-lactic acid-producing strains GT9 and GT10. The lactic acid-producing strains according to the present invention have been deposited with the China Center for Type Culture Collection (CCTCC). The deposition numbers of the D-lactic acid-producing strains GT6 and GT8 and the L-lactic acid-producing strains GT9 and GT10 are CCTCC M 20221822, CCTCC M 20221823, CCTCC M 20221824 and CCTCC M 20221825 respectively.

In some embodiments of the present invention, the constructed lactic acid-producing strain shows growth activity under a 37° C. to 70° C. temperature condition, can withstand a temperature of 37° C. to 70° C., and can produce lactic acid and/or pyruvic acid at a temperature of 37° C. to 70° C.

In some embodiments of the present invention, the constructed lactic acid-producing strain includes microorganisms of genus Bacillus and Geobacillus, and is preferably selected from Bacillus lichenformis, Bacillus coagulans, Bacillus smithii, Bacillus pallidus and Geobacillus thermoglucosidasius.

In some specific preferred embodiments, the constructed lactic acid-producing strain is Geobacillus thermoglucosidasius.

In a third aspect of the present invention, there is provided use of a lactic acid-producing strain according to the present invention in lactic acid production. In embodiments of the present invention, the lactic acid is L-lactic acid or D-lactic acid.

In a preferred embodiment of the present invention, using a lactic acid-producing strain according to the present invention, for lactic acid, can be achieved a titer of at least 80 g L⁻¹, such as 90 g L⁻¹, 100 g L⁻¹, 110 g L⁻¹, 120 g L⁻¹, 130 g L⁻¹, 140 g L⁻¹ or 150 g L⁻¹, and a yield of at least 80%, such as 90%, 91%, 92%, 93%, 94% or 95%. Preferably, chiral purity of the lactic acid is at least 99%, such as 99.3%, 99.4%, 99.5%, 99.6%, 99.7%, 99.8% or 99.9%.

In a preferred embodiment of the present invention, using a D-lactic acid-producing strain according to the present invention, for D-lactic acid, can be achieved a titer of 153.07 g L⁻¹, a yield of 93.04% and chiral purity as high as 99.63%. Using an L-lactic acid-producing strain according to the present invention, for L-lactic acid, can be achieved a titer of 151.12 g L⁻¹, a yield of 98.68% and chiral purity as high as 99.04%, or even higher.

In a fourth aspect of the present invention, there is provided a method for producing lactic acid, characterized by comprising steps of:
1) providing a lactic acid-producing strain according to the present invention;
2) at 37° C. to 70° C., culturing the strain for 6 hours to 15 hours, thereby providing a seed; and
3) at 37° C. to 70° C. in a presence of a carbon source, subjecting the seed to fermentation culture, thereby obtaining lactic acid.

In some embodiments of the present invention, the lactic acid is L-lactic acid or D-lactic acid.

In some embodiments of the present invention, the carbon source is selected from one or more of glucose, xylose, sucrose, glycerol, arabinose and mannitol.

In some embodiments of the present invention, in step 3), an inoculum volume of the seed has an OD_{620 nm} value of 0.2 to 0.8. For example, the inoculum volume is such that the OD_{620 nm} value reaches 0.2, 0.3, 0.4, 0.5, 0.6, 0.7 or 0.8.

In some embodiments of the present invention, an initial concentration of the carbon source may be 40 g/L to 100 g/L. In preferred embodiments, a fed-batch fermentation strategy is employed. In the process of fermentation, when the concentration of the carbon source drops blow 40.0 g/L, the carbon source is fed to maintain the concentration in the range of 0 to 100 g/L.

In some embodiments of the present invention, the fermentation temperature may be 37° C. to 70° C. Those skilled in the art may adjust the fermentation temperature depending on the fermentative bacterium used. Fermentation temperatures include, but are not limited to, 50° C., 55° C., 56° C., 57° C., 58° C., 59° C., 60° C., 61° C., 62° C., 63° C., 64° C., 65° C. and 70° C. Fermentation temperature ranges include, but are not limited to, 40° C. to 70° C., 50° C. to 65° C., 55° C. to 65° C. and 60° C. to 65° C.

In some embodiments of the present invention, the fermentation culture may be carried out at a pH of 6.5 to 7.5. For example, the fermentation culture may be carried out at a pH of 6.5, 6.6, 6.7, 6.8, 6.9, 7.0, 7.1, 7.2, 7.3, 7.4 or 7.5.

In some embodiments of the present invention, the fermentation culture is carried out for a period of up to 50 hours, at least 1 hour. Preferably, it is from 48 hours to 50 hours, such as 48 hours, 48.5 hours, 49 hours, 49.25 hours, 49.67 hours or 50 hours.

In some embodiments of the present invention, the fermentation culture is carried out under agitation at a speed of 50 rpm to 150 rpm.

In some embodiments of the present invention, a fermentation vessel known in the art (e.g., a fermenter) may be used to ferment the carbon source.

In some embodiments of the present invention, a fermentation product may be separated and purified. Methods known in the art may be used to separate and purify the fermentation product.

Below, by way of a few examples, the present invention will be described in greater detail. The following examples are illustrative, but not limiting. The following examples should not be construed to limit the scope of protection of the present invention. The examples of the present invention are implemented on the basis of the technical solutions of the present invention. Although specific implementation and operation details are set forth, the scope of protection of the invention is not limited to the following examples.

### EXAMPLES

Experimental methods in the following examples, for which particular conditions are not specified (e.g., PCR amplification, transformation, gene insertion, protein purification, etc.), are generally carried out in a conventional manner and conditions, for example, using the methods and conditions as taught in Molecular Cloning: A Laboratory Manual by Sambrook et al. (New York: Cold Spring Harbor Laboratory Press, 1989), or under conditions suggested by the manufacturers.

A method for genetic engineering of Geobacillus thermoglucosidasius DSM 2542 is as follows: a constructed recombinant plasmid is introduced by electroporation into Geobacillus thermoglucosidasius DSM 2542 (25 kV/cm, 10 μF, 600 Ω); the resulting transformants are cultured and then incubated on a kanamycin-resistant plate at 68° C. for 12 hours, obtaining single-crossover strains; the single-crossover strains are incubated at 60° C. for 10 hours and subcultured for 2-5 generations, followed by green fluorescence-based on-plate screening for double-crossover strains; finally, corresponding primers are used to perform PCR verification, obtaining a correct engineered strain.

A method for handling a fermentation sample is as follows: the sample is boiled at 100° C. for 10 minutes; subsequently, 2 mL of the sample is taken and added to a 100 mL volumetric flask, and 2 mL of 2M sulfuric acid is used for acid-hydrolysis for 10 minutes; the volume is then brought to 100 mL; finally, it is centrifuged at 8,000 rpm for 10 minutes, filtered using a 0.22-micron syringe membrane filter for aqueous filtration, and then can be used for subsequent detection. For chiral purity determination of lactic acid, the above prepared sample is additionally diluted 4 times and then can be used.

Conditions and a method for detection using a high-performance liquid chromatography system are as follows: the high-performance liquid chromatography system (Agilent 1260 Series, Hewlett-Packard, USA) is equipped with a Bio-Rad Aminex HPX-87H column (300×7.8 mm) and a differential refractive index detector (RID); the column temperature is 55° C.; the flow rate is 0.5 mL/min; the mobile phase is 5 mM sulfuric acid; the injection volume is 10 μL.

Conditions and a method for detecting chiral purity of lactic acid using a high-performance liquid chromatography system are as follows: the high-performance liquid chromatography system (Agilent 1260 Series, Hewlett-Packard, USA) is equipped with an SCAS Sumichiral OA-5000 column (150×4.6 mm) and a diode array detector (DAD); the column temperature is 30° C.; the flow rate is 0.8 mL/min; the mobile phase is 2 mM copper sulfate; the injection volume is 10 L; an ultraviolet absorbance wavelength is set to 254 nm.

The materials and reagents used in the examples, unless otherwise specified, can be all obtained from commercial sources.

### Example 1: Construction of D-Lactic Acid-Producing Strain According to Present Invention

1.1 Knockout of L-ldh Gene and Insertion of D-ldh Gene in Geobacillus thermoglucosidasius

By double digestion of the pUB-sfGFP plasmid with BamHI and HindIII, a linearized vector was obtained. The primer pairs LDH-Up-F/LDH-Up-R and LDH-Down-F/LDH-Down-R were used to amplify, by the polymerase chain reaction (PCR) technique, from the genome of Geobacillus thermoglucosidasius DSM 2542, the upstream and downstream homology arms respectively. The primer pair LDH-DLDH-F/LDH-DLDH-R was used to amplify by the PCR technique, from the genome of Bacillus lichenformis BJQ, the D-ldh gene. Its codon encoding the amino acid at position 101 was mutated into a glutamine-coding codon (SEQ ID NO. 2). Specifically, the mutation process was as described in Example 6. The primer pair LDH-Up-F/LDH-Down-R was used to amplify, by overlap extension polymerase chain reaction (SOE-PCR), via splicing from the templates of upstream homology arm, D-ldh gene and downstream homology arm, the LDHDLDH-UD fragment. By ligating the LDH-DLDH-UD fragment into the linearized pUB-sfGFP vector through seamless cloning using the ClonExpress Ultra One Step Cloning Kit (Vazyme, Nanjing, China), a pUB-1 plasmid was obtained. The primer pair pUBTY-F/pUBTY-R was used to verify insertion of the fragment in the MCS region.

Using the pUB-1 plasmid, through performing genetic manipulations on Geobacillus thermoglucosidasius DSM 2542, the native L-ldh gene was knocked out, and the D-ldh gene was successfully introduced at the same position, obtaining a strain GT1. The primer pair LDH-YZ-F/LDH-YZ-R was used to verify the double-crossover results.

1.2 Overexpression of pk and pvyk Genes in Strain GT1

By double digestion of the pUB-sfGFP plasmid with BamHI and HindIII, a linearized vector was obtained. The primer pairs GPFYK-Up-F/GLFYK-Up-R and GPFYAK-DOWN-F/GPFYK-DOWN-R were used to amplify, by the polymerase chain reaction (PCR) technique, from the genome of Geobacillus thermoglucosidasius DSM 2542, the upstream and downstream homology arms, respectively. The primer pair GLFYK-Pl-F/GLFYK-Pl-R was used to amplify, by the PCR technique, from the genome of Geobacillus stearothermophilus, the P_{ldh} promoter (SEQ ID NO. 7). The primer pair GLFYK-F/GPFYAK-R was used to amplify, by the PCR technique, from the genome of Bacillus coagulans H-2, the pfk and pyk genes (SEQ ID NO. 6). The primer pair GPFYK-Up-F/GPFYK-DOWN-R was used to amplify, by overlap extension polymerase chain reaction (SOE-PCR), from the templates of upstream homology arm, P_{ldh}, pfk and pyk genes and downstream homology arm, the GLFYK-UD fragment. By ligating the GLFYK-UD fragment into the linearized pUB-sfGFP vector through seamless cloning using the ClonExpress Ultra One Step Cloning Kit (Vazyme, Nanjing, China), a pUB-2 plasmid was obtained. The primer pair pUBTY-F/pUBTY-R was used to verify insertion of the fragment in the MCS region.

Using the pUB-2 plasmid, through performing genetic manipulations on Geobacillus thermoglucosidasius DSM 2542, in the strain GT1, the pfk and pyk genes were successfully overexpressed, obtaining a strain GT2. The primer pair GFYK—YZ-F/GFYK—YZ-R was used to verify the double-crossover results.

1.3 Knockout of acdh1 Gene in Strain GT2

By double digestion of the pUB-sfGFP plasmid with BamHI and HindIII, a linearized vector was obtained. The primer pairs AcDH1-Up-F/AcDH1-Up-R and AcDH1-Down-F/AcDH1-Down-R were used to amplify, by the polymerase chain reaction (PCR) technique, from the genome of Geobacillus thermoglucosidasius DSM 2542, the upstream and downstream homology arms, respectively. The primer pair AcDH1-Up-F/AcDH1-Down-R was used to amplify, by overlap extension polymerase chain reaction (SOE-PCR), from the templates of upstream and downstream homology arms, the AcDH1-UD fragment. By ligating the AcDH1-UD fragment into the linearized pUB-sfGFP vector through seamless cloning using the ClonExpress Ultra One Step Cloning Kit (Vazyme, Nanjing, China), a pUB-3 plasmid was obtained. The primer pair pUBTY-F/pUBTY-R was used to verify insertion of the fragment in the MCS region.

Using the pUB-3 plasmid, through performing genetic manipulations on Geobacillus thermoglucosidasius DSM 2542, in the strain GT2, the acdh1 gene was successfully knocked out, obtaining a strain GT3. The primer pair AcDH1-YZ-F/AcDH1-YZ-R was used to verify the double-crossover results.

1.4 Knockout of acdh2 Gene in Strain GT3

By double digestion of the pUB-sfGFP plasmid with BamHI and HindIII, a linearized vector was obtained. The primer pairs AcDH2-Up-F/AcDH2-Up-R and AcDH2-Down-F/AcDH2-Down-R were used to amplify, by the polymerase chain reaction (PCR) technique, from the genome of Geobacillus thermoglucosidasius DSM 2542, the upstream and downstream homology arms, respectively. The primer pair AcDH2-Up-F/AcDH2-Down-R was used to amplify, by overlap extension polymerase chain reaction (SOE-PCR), from the templates of upstream and downstream homology arms, the AcDH2-UD fragment. By ligating the AcDH2-UD fragment into the linearized pUB-sfGFP vector through seamless cloning using the ClonExpress Ultra One Step Cloning Kit (Vazyme, Nanjing, China), a pUB-4 plasmid was obtained. The primer pair pUBTY-F/pUBTY-R was used to verify insertion of the fragment in the MCS region.

Using the pUB-4 plasmid, through performing genetic manipulations on Geobacillus thermoglucosidasius DSM 2542, in the strain GT3, the acdh2 gene was successfully knocked out, obtaining a strain GT4. The primer pair AcDH2-YZ-F/AcDH2-YZ-R was used to verify the double-crossover results.

1.5 Knockout of aadh Gene in Strain GT4

By double digestion of the pUB-sfGFP plasmid with BamHI and HindIII, a linearized vector was obtained. The primer pairs AADH-Up-F/AADH-Up-R and AADH-Down-F/AADH-Down-R were used to amplify, by the polymerase chain reaction (PCR) technique, from the genome of Geobacillus thermoglucosidasius DSM 2542, the upstream and downstream homology arms, respectively. The primer pair AADH-Up-F/AADH-Down-R was used to amplify, by overlap extension polymerase chain reaction (SOE-PCR), from the templates of upstream and downstream homology arms, the AADH-UD fragment. By ligating the AADH-UD fragment into the linearized pUB-sfGFP vector through seamless cloning using the ClonExpress Ultra One Step Cloning Kit (Vazyme, Nanjing, China), a pUB-5 plasmid was obtained. The primer pair pUBTY-F/pUBTY-R was used to verify insertion of the fragment in the MCS region.

Using the pUB-5 plasmid, through performing genetic manipulations on the Geobacillus thermoglucosidasius DSM 2542, in the strain GT4, the aadh gene was successfully knocked out, obtaining a strain GT5. The primer pair AADH-YZ-F/AADH-YZ-R was used to verify the double-crossover results.

### 1.6 Insertion of Codon-Optimized D-ldh Gene in Strain GT5

By double digestion of the pUB-sfGFP plasmid with BamHI and HindIII, a linearized vector was obtained. The primer pairs AADH-Up-F/DLDH-Up-R and DLDH-Down-F/AADH-Down-R were used to amplify, by the polymerase chain reaction (PCR) technique, from the genome of Geobacillus thermoglucosidasius DSM 2542, the upstream and downstream homology arms, respectively. The primer pair DLDH-F/DLDH-R was used to amplify by the PCR technique the codon-optimized D-ldh gene (SEQ ID NO. 3) based on the synthesized sequence thereof. The primer pair AADH-Up-F/AADH-Down-R was used to amplify, by overlap extension polymerase chain reaction (SOE-PCR), from the templates of upstream homology arm, codon-optimized D-ldh gene and downstream homology arm, the DLDH1-UD fragment. By ligating the DLDH1-UD fragment into the linearized pUB-sfGFP vector through seamless cloning using the ClonExpress Ultra One Step Cloning Kit (Vazyme, Nanjing, China), a pUB-6 plasmid was obtained. The primer pair pUBTY-F/pUBTY-R was used to verify insertion of the fragment in the MCS region.

Using the pUB-6 plasmid, through performing genetic manipulations on Geobacillus thermoglucosidasius DSM 2542, into the strain GT5, the codon-optimized D-ldh gene was successfully inserted, obtaining a strain GT6. The primer pair AADH-YZ-F/AADH-YZ-R was used to verify the double-crossover results.

### Example 2: Inventive Construction of D-Lactic Acid-Producing Strain

Using the same methods as in Example 1, a strain GT6 was constructed, and the difference is that this example additionally includes the following manipulations.

2.1 Knockout of pflB Gene and Insertion of Codon-Optimized D-ldh Gene in Strain GT6

By double digestion of the pUB-sfGFP plasmid with BamHI and HindIII, a linearized vector was obtained. The primer pairs pflB-Up-F/pflB-Up-R and pflB-Down-F/pflB-Down-R were used to amplify, by the polymerase chain reaction (PCR) technique, from the genome of Geobacillus thermoglucosidasius DSM 2542, the upstream and downstream homology arms, respectively. The primer pair DLDH-F1/DLDH-R1 was used to amplify, by the PCR technique, based on the synthesized sequence of the codon-optimized D-ldh gene, the codon-optimized D-ldh gene (SEQ ID NO. 3). The primer pair pflB-Up-F/pflB-Down-R was used to amplify, by overlap extension polymerase chain reaction (SOE-PCR), from the templates of upstream homology arm, codon-optimized D-ldh gene and downstream homology arm, the pflB-DLDH1-UD fragment. By ligating the pflB-DLDH1-UD fragment into the linearized pUB-sfGFP vector through seamless cloning using the ClonExpress Ultra One Step Cloning Kit (Vazyme, Nanjing, China), a pUB-7 plasmid was obtained. The primer pair pUBTY-F/pUBTY-R was used to verify insertion of the fragment in the MCS region.

Using the pUB-7 plasmid, through performing genetic manipulations on Geobacillus thermoglucosidasius DSM 2542, in the strain GT6, the pflB gene was successfully knocked out and the codon-optimized D-ldh gene was inserted, obtaining a strain GT7. The primer pair pflB-YZ-F/pflB-YZ-R was used to verify the double-crossover results.

### 2.2 Knockout of pta Gene in Strain GT7

By double digestion of the pUB-sfGFP plasmid with BamHI and HindIII, a linearized vector was obtained. The primer pairs pta-Up-F/pta-Up-R1 and pta-Down-F1/pta-Down-R were used to amplify, by the polymerase chain reaction (PCR) technique, from the genome of Geobacillus thermoglucosidasius DSM 2542, the upstream and downstream homology arms, respectively. The primer pair pta-Up-F/pta-Down-R was used to amplify, by overlap extension polymerase chain reaction (SOE-PCR), from the templates of upstream and downstream homology arms, the pta-UD fragment. By ligating the pta-UD fragment into the linearized pUB-sfGFP vector through seamless cloning using the ClonExpress Ultra One Step Cloning Kit (Vazyme, Nanjing, China), a pUB-8 plasmid was obtained. The primer pair pUBTY-F/pUBTY-R was used to verify insertion of the fragment in the MCS region.

Using the pUB-8 plasmid, through performing genetic manipulations on Geobacillus thermoglucosidasius DSM 2542, in the strain GT7, the pta gene was successfully knocked out, obtaining a strain GT8. The primer pair pta-YZ-F/pta-YZ-R was used to verify the double-crossover results.

### Example 3: Inventive Construction of L-Lactic Acid-Producing Strain

Using the same methods as in Example 1, a strain GT6 was constructed, and the difference is that this example additionally includes the following manipulations.

### 3.1 Knockout of D-ldh Gene, Insertion of L-ldh Gene and Knockout of Codon-Optimized D-ldh Gene in Strain GT6

By double digestion of the pUB-sfGFP plasmid with BamHI and HindIII, a linearized vector was obtained. The primer pair LDH-Up-F/LDH-Down-R was used to amplify, by the polymerase chain reaction (PCR) technique, from the genome of Geobacillus thermoglucosidasius DSM 2542, the LLDH-UD fragment. By ligating the LLDH-UD fragment into the linearized pUB-sfGFP vector through seamless cloning using the ClonExpress Ultra One Step Cloning Kit (Vazyme, Nanjing, China), a pUB-9 plasmid was obtained. The primer pair pUBTY-F/pUBTY-R was used to verify insertion of the fragment in the MCS region.

Using the pUB-9 and pUB-5 plasmids, through performing genetic manipulations on Geobacillus thermoglucosidasius DSM 2542, in the strain GT6, the D-ldh gene was successfully knocked out, the L-ldh gene (SEQ ID No. 8) was inserted and the codon-optimized D-ldh gene was knocked out, obtaining a strain GT9. The primer pairs LDH-YZ-F/LDH-YZ-R and AADH-YZ-F/AADH-YZ-R were used to verify the double-crossover results.

3.2 Insertion of L-ldh Gene Derived from Bacillus coagulans H-2 in Strain GT9

By double digestion of the pUB-sfGFP plasmid with BamHI and HindIII, a linearized vector was obtained. The primer pairs AADH-Up-F/LLDH-Up-R1 and LLDH-Down-F1/AADH-Down-R were used to amplify by the polymerase chain reaction (PCR) technique, from the genome of Geobacillus thermoglucosidasius DSM 2542, the upstream and downstream homology arms, respectively. The primer pair LLDH-F1/LLDH-R1 was used to amplify, by the PCR technique, from the genome of Bacillus coagulans H-2, the L-ldh gene (SEQ ID NO. 5). The primer pair AADH-Up-F/AADH-Down-R was used to amplify, by overlap extension polymerase chain reaction (SOE-PCR), from the templates of upstream homology arm, L-ldh gene derived from Bacillus coagulans H-2 and downstream homology arm, the LLDH1-UD fragment. By ligating the LLDH1-UD fragment into the linearized pUB-sfGFP vector through seamless cloning using the ClonExpress Ultra One Step Cloning Kit (Vazyme, Nanjing, China), a pUB-10 plasmid was obtained. The primer pair pUBTY-F/pUBTY-R was used to verify insertion of the fragment in the MCS region.

Using the pUB-10 plasmid, through performing genetic manipulations on Geobacillus thermoglucosidasius DSM 2542, into the strain GT9, the L-ldh gene derived from Bacillus coagulans H-2 was successfully inserted, obtaining a strain GT10. The primer pair AADH-YZ-F/AADH-YZ-R was used to verify the double-crossover results.

### Example 4: D-Lactic Acid Production According to Present Invention

D-lactic acid-producing strains constructed in accordance with the present invention were subjected to, in a 5-L fermenter at 60° C., fed-batch fermentation.

Strain culture conditions: 37° C., 200 rpm, a fermentation culture period was up to 50 hours.

A strain culture medium contained the following components: 10 g L⁻¹ NaCl, 5 g L⁻¹ yeast extract, 10 g L⁻¹ peptone.

After the cultured strains were incubated in a seed culture medium for fermentation overnight, they were used to be inoculated into a fermentation medium for fermentation production.

The seed culture medium for fermentation contained the following components: 5 g L⁻¹ soya peptone, 15 g L⁻¹ peptone, 5 g L⁻¹ NaCl.

The fermentation medium contained the following components: 5 g L⁻¹ yeast extract, 3 g L⁻¹ Na₂HPO₄, 3 g L⁻¹ KH₂PO₄, 1 g L⁻¹ NH₄Cl, 0.48 g L⁻¹ MgSO₄, 0.5 g L⁻¹ NaCl, 0.42 g L⁻¹ citric acid, 0.028 g L⁻¹ FeSO₄·7H₂O, 0.01 g L⁻¹ thiamin, Trace Metal Mix (4.4 mg L⁻¹ NiSO₄·6H₂O, 2.86 mg L⁻¹ H₃BO₃, 1.81 mg L⁻¹ MnCl₂₋₄H₂O, 0.39 mg L⁻¹ Na₂MoO₄·2H₂O, 0.222 mg L⁻¹ ZnSO₄-7H₂O, 0.079 mg L⁻¹ CuSO₄-5H₂O, 0.049 mg L⁻¹ Co(NO₃)₂₋₆H₂O), 3.1 mg L⁻¹ biotin, 0.1 g L⁻¹ betaine.

The seed for fermentation was inoculated at an inoculum volume of 0.3% (v/v) to 10% (v/v).

4.1 D-Lactic Acid Production by Strain GT1 from Glucose as Carbon Source

The strain GT1 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, a D-lactic acid titer was 37.46 g L⁻¹, a D-lactic acid yield was 5²0.40%, a D-lactic acid titer production rate was 0.780 g L⁻¹ h⁻¹.

4.2 D-Lactic Acid Production by Strain GT2 from Glucose as Carbon Source

The strain GT2 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, a D-lactic acid titer was 42.96 g L⁻¹, a D-lactic acid yield was 49.50%, a D-lactic acid production rate was 0.895 g L⁻¹ h⁻¹.

4.3 D-Lactic Acid Production by Strain GT3 from Glucose as Carbon Source

The strain GT3 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, a D-lactic acid titer was 33.43 g L⁻¹, a D-lactic acid yield was 58.48%, a D-lactic acid production rate was 0.696 g L⁻¹ h⁻¹.

4.4 D-Lactic Acid Production by Strain GT4 from Glucose as Carbon Source

The strain GT4 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 49 hours; samples were taken and subjected to high-performance liquid chromatography detection; according to the results of detection, after fermentation for 49 hours, in the fermentation broth, a D-lactic acid titer was 27.42 g L⁻¹, a D-lactic acid yield was 49.66%, a D-lactic acid production rate was 0.560 g L⁻¹ h⁻¹.

4.5 D-Lactic Acid Production by Strain GT5 from Glucose as Carbon Source

The strain GT5 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, a D-lactic acid titer was 57.38 g L⁻¹, a D-lactic acid yield was 76.79%, a D-lactic acid production rate was 1.148 g L⁻¹ h⁻¹.

4.6 D-Lactic Acid Production by Strain GT6 from Glucose as Carbon Source

The strain GT6 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48.5 hours; samples were taken and subjected to high-performance liquid chromatography detection; according to the results of detection, after fermentation for 48.5 hours, in the fermentation broth, a D-lactic acid titer was 86.06 g L⁻¹, a D-lactic acid yield was 92.05%, a D-lactic acid production rate was 1.774 g L⁻¹ h⁻¹.

4.7 D-Lactic Acid Production by Strain GT6 from Glucose as Carbon Source

The strain GT6 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 100 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, a D-lactic acid titer was 153.07 g L⁻¹, a D-lactic acid yield was 93.04%, a D-lactic acid production rate was 3.189 g L⁻¹ h⁻¹, chiral purity ofD-lactic acid was 99.63%.

4.8 D-Lactic Acid Production by Strain GT6 from Xylose as Carbon Source

The strain GT6 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial xylose concentration was 40 g L⁻¹, in the process of fermentation, when the xylose concentration was 0.0 g/L to 40.0 g/L, xylose was added to the fermenter to maintain the xylose concentration within the range of 0 to 100.0 g/L, a fermentation period was 48 hours; samples were taken and subjected to high-performance liquid chromatography detection; according to the results of detection, after fermentation for 48 hours, in the fermentation broth, a D-lactic acid titer was 30.81 g L⁻¹, a D-lactic acid yield was 88.31%, a D-lactic acid production rate was 0.642 g L⁻¹ h⁻¹.

4.9 D-Lactic Acid Production by Strain GT7 from Glucose as Carbon Source

The strain GT7 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, a D-lactic acid titer was 93.82 g L⁻¹, a D-lactic acid yield was 93.27%, a D-lactic acid production rate was 1.955 g L⁻¹ h⁻¹.

4.10 D-Lactic Acid Production by Strain GT8 from Glucose as Carbon Source

The strain GT8 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, a D-lactic acid titer was 88.82 g L⁻¹, a D-lactic acid yield was 92.21%, a D-lactic acid production rate was 1.850 g L⁻¹ h⁻¹.

### Example 5: Production of L-Lactic Acid According to Present Invention

L-lactic acid-producing strains constructed in accordance with the present invention were subjected to, in a 5-L fermenter at 60° C., fed-batch fermentation.

Components of the seed culture medium, seed culture medium for fermentation and fermentation medium were the same as in Example 4.

5.1 L-Lactic Acid Production by Strain GT9 from Glucose as Carbon Source

The strain GT9 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, an L-lactic acid titer was 92.34 g L⁻¹, an L-lactic acid yield was 95.29%, an L-lactic acid production rate was 1.924 g L⁻¹ h⁻¹, and chiral purity of L-lactic acid was 99.46%.

5.2 L-Lactic Acid Production by Strain GT10 from Glucose as Carbon Source

The strain GT10 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, an L-lactic acid titer was 94.15 g L⁻¹, an L-lactic acid yield was 91.52%, an L-lactic acid production rate was 1.961 g L⁻¹ h⁻¹, and chiral purity of L-lactic acid was 99.50%.

5.3 L-Lactic Acid Production by Strain GT10 from Glucose as Carbon Source

The strain GT10 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 100 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, an L-lactic acid titer was 151.12 g L⁻¹, an L-lactic acid yield was 98.68%, an L-lactic acid production rate was 3.148 g L⁻¹ h⁻¹, and chiral purity of L-lactic acid was 99.04%.

### Comparative Example 1: Fed-Batch Fermentation by Geobacillus thermoglucosidasius DSM 2542 in 5-L Fermenter at 60° C.

Non-genetically engineered Geobacillus thermoglucosidasius DSM 2542 was used to produce L-lactic acid. In this example, components of the seed culture medium, seed culture medium for fermentation and fermentation medium were the same as in Example 5.

Geobacillus thermoglucosidasius DSM 2542 was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 49.67 hours; samples were taken and subjected to high-performance liquid chromatography detection; according to the results of detection, after fermentation for 49.67 hours, in the fermentation broth, an L-lactic acid titer was 69.17 g L⁻¹, an L-lactic acid yield was 88.14%, an L-lactic acid production rate was 1.393 g L⁻¹ h⁻¹.

### Comparative Example 2: Fed-Batch Fermentation by Bacillus smithii in 5-L Fermenter at 60° C.

Non-genetically engineered Bacillus smithii was used to produce L-lactic acid, as well as the same seed culture medium. In this example, components of the seed culture medium, seed culture medium for fermentation and fermentation medium were the same as in Example 5.

Bacillus smithii was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 49.25 hours; samples were taken and subjected to high-performance liquid chromatography detection; according to the results of detection, after fermentation for 49.25 hours, in the fermentation broth, an L-lactic acid titer was 43.50 g L⁻¹, an L-lactic acid yield was 83.60%, an L-lactic acid production rate was 0.883 g L⁻¹ h⁻¹.

### Comparative Example 3: Fed-Batch Fermentation by Bacillus pallidus in 5-L Fermenter at 60° C.

Non-genetically engineered Bacillus pallidus was used to produce L-lactic acid, as well as the same seed culture medium. In this example, components of the seed culture medium, seed culture medium for fermentation and fermentation medium were the same as in Example 5.

Bacillus pallidus was cultured in the seed culture medium for fermentation at 60° C. and 200 rpm overnight, the seed was inoculated into the fermentation medium at 10% (v/v) and subjected to fermentation culture; fermentation conditions: 25% (w/v) Ca(OH)₂ was used to maintain a pH at 7.0, an agitation speed was 80 rpm, a culture temperature was 60° C., an initial glucose concentration was 40 g L⁻¹, in the process of fermentation, when the glucose concentration was 0.0 g/L to 40.0 g/L, glucose was added to the fermenter to maintain the glucose concentration within the range of 0 to 100.0 g/L, a fermentation period was 48 hours; samples were taken and subjected to high-performance liquid chromatography detection; according to the results of detection, after fermentation for 48 hours, in the fermentation broth, an L-lactic acid titer was 13.80 g L⁻¹, an L-lactic acid production rate was 0.288 g L⁻¹ h⁻¹.

The results of D-lactic acid production in Example 4 are summarized in Table 2 below.

TABLE 2
D-Lactic Acid Production

| | | | | Production | |
|---|---|---|---|---|---|
| | Carbon Source | Titer of | | Rate of | Chiral |
| | and Initial | D-Lactic | Yield of | D-Lactic | Purity of |
| | Concentration | Acid | D-Lactic | Acid | D-lactic |
| Strain | (g L⁻¹) | (g L⁻¹) | Acid (%) | (g L⁻¹ h⁻¹) | acid (%) |
| GT1 | glucose 40 | 37.46 | 52.40 | 0.780 | — |
| GT2 | glucose 40 | 42.96 | 49.50 | 0.895 | — |
| GT3 | glucose 40 | 33.43 | 58.48 | 0.696 | — |
| GT4 | glucose 40 | 27.42 | 49.66 | 0.560 | — |
| GT5 | glucose 40 | 57.38 | 76.79 | 1.148 | — |
| GT6 | glucose 40 | 86.06 | 92.05 | 1.774 | — |
| GT6 | glucose 100 | 153.07 | 93.04 | 3.189 | 99.63 |
| GT6 | xylose 40 | 30.81 | 88.31 | 0.642 | — |
| GT7 | glucose 40 | 93.82 | 93.27 | 1.955 | — |
| GT8 | glucose 40 | 88.82 | 92.21 | 1.850 | — |

“—” means not determined.

As can be seen from the results of Table 2, after introducing and optimizing the D-lactic acid synthesis pathway and inhibiting the by-product synthesis pathways through the efficient D-lactic acid-producing strain construction stratery of the present invention, the obtained strain GT6, when used for D-lactic acid production by fermentation of glucose as a carbon source, can achieve a D-lactic acid titer of up to 153.07 g L⁻¹, a D-lactic acid yield of up to 93.04%, a D-lactic acid production rate of up to 3.189 g L⁻¹ h⁻¹, and chiral purity of D-lactic acid was up to 99.63%.

Under the same production conditions, compared with the strain GT1 with only the introduction of the D-lactic acid synthesis pathway, GT6 achieved a titer and a production rate each more than 4 times that of GT1, and a D-lactic acid yield increased by about 78%. Compared with the strain GT2 with only the introduction and partially optimization of the D-lactic acid synthesis pathway and the strains GT3, GT4 and GT5 with only the introduction and partially optimization of the D-lactic acid synthesis pathway and partially inhibition of by-product synthesis pathways, GT6 that has experienced all the three engineering pathways exhibited significantly enhanced ability to produce D-lactic acid and is remarkably improved in terms of D-lactic acid titer, D-lactic acid yield and D-lactic acid production rate.

Additionally, the efficient D-lactic acid-producing strains according to the present invention can use different carbon sources. For example, when using xylose as a carbon source, GT6 can also obtain a D-lactic acid yield of 88.31%.

On the basis of GT6, the D-lactic acid synthesis pathway is further optimized and by-product synthesis pathways are inhibited, obtaining the strains GT7 and GT8. When using glucose as a carbon source, GT7 and GT8 maintain a high D-lactic acid titer of 88.82 g L⁻¹ or more and a high D-lactic acid yield of 92.21% or above.

The results of L-lactic acid production in Example 5 and Comparative Examples 1 to 3 are summarized in Table 3 below.

TABLE 3
L-Lactic Acid Production

| | Carbon Source | Titer of | | Production | Chiral |
|---|---|---|---|---|---|
| | and Initial | L-Lactic | Yield of | Rate of | Purity of |
| | Concentration | Acid | L-Lactic | L-Lactic Acid | L-lactic |
| Strain | (g L⁻¹) | (g L⁻¹) | Acid (%) | (g L⁻¹ h⁻¹) | acid (%) |
| Inventive GT9 | glucose 40 | 92.34 | 95.29 | 1.924 | 99.46 |
| Inventive GT10 | glucose 40 | 94.15 | 91.52 | 1.961 | 99.50 |
| Inventive GT10 | glucose 100 | 151.12 | 98.68 | 3.148 | 99.04 |
| Comparative | glucose 40 | 69.17 | 88.14 | 1.393 | — |
| Example 1 | | | | | |
| (Geobacillus | | | | | |
| thermoglucosidasius | | | | | |
| DSM 2542) | | | | | |
| Comparative | glucose 40 | 43.50 | 83.60 | 0.883 | — |
| Example 2 (Bacillus | | | | | |
| smithii) | | | | | |
| Comparative | glucose 40 | 13.80 | — | 0.288 | — |
| Example 3 (Bacillus | | | | | |
| pallidus) | | | | | |

“—” means not determined.

As can be seen from Table 3, under the same production conditions, or even within a shorter fermentation time, the engineered strains GT9 and GT10 according to the present invention can obtain an L-lactic acid titer of 92.34 g L⁻¹ or above, an L-lactic acid yield of up to 95.29% and an L-lactic acid production rate higher than 1.924 g L⁻¹ h⁻¹, and chiral purity of L-lactic acid is 99.46% or higher. When an initial glucose concentration reaches 100 g L⁻¹, the engineered strain GT10 according to the present invention can obtain an L-lactic acid titer of up to 151.12 g L⁻¹, an L-lactic acid yield of up to 98.68% and an L-lactic acid production rate of up to 3.148 g L⁻¹ h⁻¹, and chiral purity of L-lactic acid is up to 99.04%. In contrast, the non-engineered starting Geobacillus thermoglucosidasius DSM 2542 strain only obtains an L-lactic acid titer of 69.17 g L⁻¹, an L-lactic acid yield of 88.14% and an L-lactic acid production rate of 1.393 g L⁻¹ h⁻¹; the non-engineered Bacillus smithii strain produced L-lactic acid can only obtain an L-lactic acid titer of 43.50 g L⁻¹, an L-lactic acid yield of 83.60% and an L-lactic acid production rate of only 0.883 g L⁻¹ h⁻¹; with the non-engineered Bacillus pallidus strain, an obtained L-lactic acid titer is as low as 13.80 g L⁻¹, an L-lactic acid production rate is only 0.288 g L⁻¹ h⁻¹. These indicate that, compared with the starting strains, the L-lactic acid-producing strains constructed through combined manipulations according to the present invention significantly increase the L-lactic acid titer and L-lactic acid production rate in L-lactic acid production. Moreover, compared with other non-engineered strains, the L-lactic acid-producing strains of the present invention are also advantageous in producing L-lactic acid with higher chiral purity and higher efficiency.

### Example 6: Directed Engineering, Expression and Purification of D-Lactate Dehydrogenase

The amino acid sequences and nucleotide sequences involved in Example 6 are as follows:

(D-LDH^{P101G}):
SEQ ID No. 71
MKVIFFSMHPYEEEFLGPILPSDWDVEMTPDFLDETTVEKAKGAQVVSLF
VSDKADGPVLEALHSYGVGLLALRSAGYDHIDIETAKRLGIKVVNVPAYS
GHAIADHTLAIMLALIRRLHRAHDKVRLGDFDLDGLMGFDLNGKVAGVIG
LGKIGRLVATRLKAFGCKVLGYDPYIQPEIVENVDLDTLITQADIISIHC
PLTRENFHMFNEETFKRMKPGAILVNTARGGLIDTKALLEALKSGKLGGA
ALDVYEYERGLFFKNHQKEGIKDPYLAQLLGLANVVLTGHQAFLTREAVK
NIEETTVENILEWQKNPQAKLKNEI
(D-LDH):
SEQ ID No. 72
MKVIFFSMHPYEEEFLGPILPSDWDVEMTPDFLDETTVEKAKGAQVVSLF
VSDKADGPVLEALHSYGVGLLALRSAGYDHIDIETAKRLGIKVVNVPAYS
PHAIADHTLAIMLALIRRLHRAHDKVRLGDFDLDGLMGFDLNGKVAGVIG
LGKIGRLVATRLKAFGCKVLGYDPYIQPEIVENVDLDTLITQADIISIHC
PLTRENFHMFNEETFKRMKPGAILVNTARGGLIDTKALLEALKSGKLGGA
ALDVYEYERGLFFKNHQKEGIKDPYLAQLLGLANVVLTGHQAFLTREAVK
NIEETTVENILEWQKNPQAKLKNEI
(codon-optimized sequence of D-LDH coding
sequence for Bacillus):
SEQ ID No. 73
ATGAAAGTAATTTTTTTTTCTATGCACCCGTATGAAGAGGAATTTCTGGG
TCCGATTCTGCCGTCTGACTGGGACGTAGAAATGACCCCGGACTTTCTGG
ACGAAACCACCGTGGAAAAGGCTAAAGGTGCCCAGGTAGTAAGCCTGTTT
GTTTCTGACAAAGCTGATGGTCCGGTACTGGAAGCGCTGCATTCTTACGG
TGTGGGCCTGCTGGCCCTGCGTTCTGCTGGCTATGATCACATCGATATTG
AGACCGCAAAACGCCTGGGTATCAAAGTAGTTAACGTGCCAGCCTATTCT
CCGCACGCTATCGCTGACCATACTCTGGCTATCATGCTGGCTCTGATTCG
TCGTCTGCACCGTGCCCATGATAAAGTGCGCCTGGGTGATTTTGATCTGG
ATGGTCTGATGGGCTTTGATCTGAACGGCAAAGTTGCTGGTGTAATTGGT
CTGGGTAAAATCGGTCGCCTGGTAGCTACCCGCCTGAAAGCGTTTGGTTG
CAAAGTTCTGGGCTATGATCCATACATTCAGCCGGAAATCGTAGAAAACG
TTGATCTGGATACCCTGATCACTCAGGCTGATATCATTTCTATTCATTGT
CCGCTGACCCGTGAAAACTTTCATATGTTTAACGAAGAGACTTTTAAGCG
TATGAAACCGGGTGCTATTCTGGTTAACACCGCGCGTGGTGGTCTGATCG
ATACCAAGGCCCTGCTGGAGGCCCTGAAGTCTGGTAAACTGGGCGGCGCA
GCCCTGGATGTGTATGAATATGAACGTGGCCTGTTTTTTAAAAACCACCA
AAAAGAAGGTATCAAAGACCCGTATCTGGCCCAGCTGCTGGGTCTGGCCA
ACGTAGTGCTGACCGGTCATCAGGCCTTTCTGACCCGTGAGGCTGTAAAA
AACATCGAAGAAACTACCGTAGAAAACATTCTGGAATGGCAAAAGAACCC
GCAGGCAAAGCTGAAAAACGAAATCTAA

The sequences of the primers used in Example 6 are as follows:

ldh-F:
(SEQ ID No. 74)
CGCGGATCCGATGAAAGTAATTTTTTTTTCTATGCAC
ldh-R:
(SEQ ID No. 75)
CCCAAGCTTTTAGATTTCGTTTTTCAGCTTTG
ldh1-R:
(SEQ ID No. 76)
TAGCGTGCTGAGAATAGG
ldh1-F:
(SEQ ID No. 77)
CCTATTCTCAGCACGCTA
ldh2-R:
(SEQ ID No. 78)
TAGCGTGCCCAGAATAGG
ldh2-F:
(SEQ ID No. 79)
CCTATTCTGGGCACGCTA
ldh3-R:
(SEQ ID No. 80)
TAGCGTGGTTAGAATAGGCTG
ldh3-F:
(SEQ ID NO. 81)
CAGCCTATTCTAACCACGCTA

In this example, prepared were wild-type D-lactate dehydrogenase D-LDH, D-lactate dehydrogenase mutants D-LDH^{P101Q} and D-LDH^{P101N} and a comparative D-lactate dehydrogenase mutant D-LDH^{P101G}. Wherein, the amino acid sequence of D-LDH is as shown in SEQ ID No. 72; D-LDH^{P101Q} is from a proline-to-glutamine mutation of D-LDH at position 101, and its amino acid sequence is as shown in SEQ ID No. 9; D-LDH^{P101N} is from a proline-to-asparagine mutation of D-LDH at position 101, and its amino acid sequence is as shown in SEQ ID No. 10; D-LDH^{P101G} is from a proline-to-glycine mutation of D-LDH at position 101, and its amino acid sequence is as shown in SEQ ID No. 71.

A specific preparation process is as follows:

The primer pair ldh-F (BamHI)/ldh-R (HindIII) was used to amplify, by the polymerase chain reaction (PCR) technique, from the genome of Bacillus lichenformis BJQ, the original D-ldh gene, and the BamHI and HindIII restriction sites were introduced. The vector pETDuet-1 was double digested with BamHI and HindIII, and the D-ldh gene was then cloned into the BamHI-HindIII site of the pETDuet-1 vector's multiple cloning site (MCS) I, producing pETDuet-ldh for D-LDH expression and purification.

The primer pairs ldh-F/ldh1-R and ldh1F/ldh-R were used to amplify, from the template of D-ldh gene, by the overlap extension polymerase chain reaction (SOE-PCR) technique, obtaining the mutated gene D-ldh1 (CCG/CAG; encoding the protein D-LDH^{P101Q}), and the BamHI and HindIII restriction sites were introduced. The vector pETDuet-1 was double digested with BamHI and HindIII, and the D-ldhI gene was then cloned into the BamHI-HindIII site of the MCS I of the pETDuet-1 vector, producing pETDuet-ldhI for D-LDH^{P101Q} expression and purification.

The primer pairs ldh-F/ldh2-R and ldh2-F/ldh-R were used to amplify, from the template of D-ldh gene, by the overlap extension polymerase chain reaction (SOE-PCR) technique, obtaining the mutated gene D-ldh2 (CCG/GGG; encoding the protein D-LDH^{P101G}), and the BamHI and HindIII restriction sites were introduced. The vector pETDuet-1 was double digested with BamHI and HindIII, and the D-ldh2 gene was then cloned into the BamHI-HindIII site of the MCS I of the pETDuet-1 vector, producing pETDuet-ldh2 for D-LDH^{P101G} expression and purification.

The primer pairs ldh-F/ldh3-R and ldh3-F/ldh-R were used to amplify, from the template of D-ldh gene, by the overlap extension polymerase chain reaction (SOE-PCR) technique, obtaining the mutated gene D-ldh3 (CCG/AAC; encoding the protein D-LDH^{P101N}), and the BamHI and HindIII restriction sites were introduced. The vector pETDuet-1 was double digested with BamHI and HindIII, and the D-ldh3 gene was then cloned into the BamHI-HindIII site of the MCS I of the pETDuet-1 vector, producing pETDuet-ldh3 for D-LDH^{P101N} expression and purification.

pETDuet-ldh, pETDuet-ldh1, pETDuet-ldh2 and pETDuet-ldh3 were separately introduced into Escherichia coli BL21 (DE3) strains (purchased from Novagen), obtaining four recombinant Escherichia coli strains; the four recombinant Escherichia coli strains were then each cultured in a seed culture medium (containing 10 g L⁻¹ NaCl, 5 g L⁻¹ yeast extract and 10 g L⁻¹ peptone) supplemented with ampicillin (100 g mL⁻¹) at 37° C. and 200 rpm. When the cell density OD_{620 nm} reached 0.6-0.8, through adding 0.4 mM isopropyl-β-D-thiogalactoside and continuing incubation at 16° C. for about 20 hours, recombinant D-LDH expression was induced. The collected cells were washed twice with a 10 mM phosphate buffer, and the cells were then resuspended in an equilibration buffer (25 mM tris(hydroxymethyl)aminomethane hydrochloride (Tris-HCl), 500 mM NaCl, 25 mM imidazole, pH 8.0), and disrupted at 4° C. by high pressure treatment. The lysed cells were centrifuged at 4° C. and 12,000 rpm for 40 minutes, and the supernatant was then used for subsequent purification.

A nickel column method was used to purify D-lactate dehydrogenase and the mutants. Specifically, Ni²⁺-NTA columns were used to purify, via affinity chromatography, from the crude extracts obtained as described above, D-lactate dehydrogenase and the mutants. The columns had been each equilibrated with 30 mL of the equilibration buffer, and with a wash buffer (25 mM Tris-HCl, 500 mM NaCl, 80 mM imidazole, pH 8.0) and an eluting/dissolving buffer (25 mM Tris-HCl, 500 mM NaCl, 300 mM imidazole, pH 8.0), D-LDH was washed and eluted; then, the target protein solutions were transferred to Amicon Ultra-15 30 K centrifugal ultrafilters for further protein concentration, and finally they were desalted by gel filtration on an AKTA Purifier equipped with a Superdex 200 10/300 GL column. The gel-filtration buffer was a 10 mM phosphate buffer (pH 7.0). Through sodium dodecyl-sulfate polyacrylamide gel electrophoresis (SDS-PAGE), the expressed and purified enzymes were measured. All the purification processes were carried out at 4° C. to ensure activity of the enzymes, and the purified enzymes were quickly frozen in liquid nitrogen and then stored at −80° C. for investigative experimentation.

According to the results of SDS-PAGE and gel filtration chromatography on the ÄKTA Purifier, the molecular weights of D-LDH, D-LDH^{P101Q}, D-LDH^{P101N} and D-LDH^{P101G} were all obtained as to be approximately 35 kD, and all of them were of tetramer structures.

### Example 7: Enzymatic Kinetics of D-Lactate Dehydrogenase and Mutants

Enzyme catalysis testing method for D-lactate dehydrogenase and the mutants: a reaction mixture had a total volume of 0.8 mL, in which were contained: 10 mM phosphate buffer (pH 7.0), an appropriate amount (0.0001 mg/L to 0.0035 mg/L) of purified D-lactate dehydrogenase or a mutant thereof, 0.25 mM NADH, sodium pyruvate at a cencontration of 0.01-0.4 mM or sodium pyruvate at a cencontration of 0.625 mM, NADH at a cencontration of 0.005-0.2 mM. In this testing method, wild-type D-lactate dehydrogenase and the mutants use the same reaction conditions. The testing temperature was 50° C. Based on the initial rate of change in absorbance at 340 nm due to the oxidation of NADH (ε340=6,220 M⁻¹ cm⁻¹), catalytic performance of D-lactate dehydrogenase and the mutants was measured.

According to the results of the above tests, catalytic activity (k_{cat}), specific enzyme activity and catalytic efficiency were calculated. The calculation method is the same as a method commonly used in the art.

Through the above tests, enzymatic kinetics of D-lactate dehydrogenase and the mutants were characterized, and the test data is summarized in Table 4 below.

TABLE 4
Enzymatic Kinetics of D-Lactate Dehydrogenase and Mutants

| | Enzymatic | | D-LDH^{P101Q} | D-LDH^{P101N} | D-LDH^{P101G} |
|---|---|---|---|---|---|
| Substrate | Kinetics | D-LDH | (inventive) | (inventive) | (comparative) |
| Sodium | k_{cat} (s⁻¹) | 6.67 ± 0.16 | 145.00 ± 4.62 | 106.60 ± 2.82 | 12.56 ± 0.27 |
| Pyruvate | Specific | 11.11 ± 0.26 | 241.70 ± 7.71 | 177.70 ± 4.70 | 20.94 ± 0.46 |
| | Enzymatic | | | | |
| | Activity (U | | | | |
| | mg⁻¹) | | | | |
| | k_{cat}/Kₘ (s⁻¹ | 2.14 × 10⁵ | 1.07 × 10⁶ | 7.47 × 10⁵ | 1.81 × 10⁵ |
| | M⁻¹) | | | | |
| NADH | k_{cat} (s⁻¹) | 13.21 ± 0.17 | 145.10 ± 1.71 | 102.60 ± 1.23 | 22.60 ± 0.50 |
| | Specific | 22.02 ± 0.28 | 241.90 ± 2.85 | 171.00 ± 2.06 | 37.67 ± 0.83 |
| | Enzymatic | | | | |
| | Activity (U | | | | |
| | mg⁻¹) | | | | |
| | k_{cat}/Kₘ (s⁻¹ | 9.38 × 10⁵ | 5.44 × 10⁶ | 7.50 × 10⁶ | 7.66 × 10⁵ |
| | M⁻¹) | | | | |

As can be seen from the results of Table 4, when the mutant D-LDH^{P101Q} is obtained by a proline-to-glutamine mutation at position 101 of D-LDH, catalytic performance is significantly improved. Specifically, in the case of sodium pyruvate being a substrate, catalytic activity (k_{cat}) of D-LDH^{P101Q} is about 22 times that of D-LDH, specific enzyme activity is about 22 times that of D-LDH, and catalytic efficiency (k_{cat}/Kₘ) is about 5 times that of D-LDH. In the case of NADH being a substrate, catalytic activity (k_{cat}) of D-LDH^{P101Q} is about 11 times that of D-LDH, specific enzyme activity is about 11 times that of D-LDH, and catalytic efficiency (k_{cat}/Kₘ) is about 6 times that of D-LDH.

Similarly, when the mutant D-LDH^{P101N} is obtained by a proline-to-asparagine mutation at position 101 of D-LDH, catalytic performance is significantly improved. Specifically, in the case of sodium pyruvate being a substrate, catalytic activity (k_{cat}) of D-LDH^{P101N} is about 16 times that of D-LDH, specific enzyme activity is about 16 times that of D-LDH, and catalytic efficiency (k_{cat}/Kₘ) is about 3.5 times that of D-LDH. In the case of NADH being a substrate, catalytic activity (k_{cat}) of D-LDH^{P101N} is about 8 times that of D-LDH, specific enzyme activity is about 8 times that of D-LDH, and catalytic efficiency (k_{cat}/Kₘ) is about 8 times that of D-LDH.

However, when the mutant D-LDH^{P101G} is obtained by a proline-to-glycine mutation at position 101 of D-LDH, catalytic activity (k_{cat}) and specific enzyme activity are only slightly improved over D-LDH (less than twice), and catalytic efficiency (k_{cat}/Kₘ) is even lower than that of D-LDH.

This indicates that the mutation position and the species of target amino acids for mutation selected by the present invention can make the obtained D-lactate dehydrogenase mutants' catalytic performance significantly improved. This is a result of creative effort. However, when the amino acid is mutated into glycine, ideal catalytic performance improving results are not obtained.

### Example 8: Thermostability of D-Lactate Dehydrogenase and Mutants

Thermostability testing method for D-lactate dehydrogenase or the mutant: D-lactate dehydrogenase and the mutants were subjected to water bath at 50° C., within the time period from 0 to 70 hours, at different time points, samples were taken and used for enzyme activity testing.

Specific method: 0.004 mg/L of D-lactate dehydrogenase or the mutant (ensuring final concentrations of the different D-lactate dehydrogenase and mutants in the reaction system were consistent) was added to a reaction system, the reaction mixture had a total volume of 0.8 mL, in which were contained 10 mM PBS (pH 7.0), 0.25 mM NADH, 0.625 mM sodium pyruvate, and when at 50° C., testing was performed. The enzyme activity measured at 0 hours of incubation of D-lactate dehydrogenase or the mutant was taken as 100% (initial enzyme activity), and the enzyme activity measured at other time points was represented by a percentage relative to the initial enzyme activity.

Thermostability data of D-lactate dehydrogenase and the mutants is summarized in Table 5 below.

TABLE 5
Thermostability of D-lactate Dehydrogenase and Mutants at 50° C.

| Incubation | | D-LDH^{P101Q} | D-LDH^{P101N} | D-LDH^{P101G} |
|---|---|---|---|---|
| Time at 50° C. | D-LDH | (inventive) | (inventive) | (comparative) |
| 0 hours | 100% | 100% | 100% | 100% |
| 0-9 hours | gradual increase | slight increase | relatively high | gradual increase |
| 9-44 hours | up to 1194% | slight decrease | relatively high | up to 288% |
| 44-67 hours | slight decrease | slight decrease | relatively high | gradual decrease |
| 67 hours | 968% | 60.53% | 100.73% | 89.07% |

As can be seen from the results of Table 5, for the D-lactate dehydrogenase mutant D-LDH^{P101Q} according to the present invention, thermostability when at 50° C. is very good, the D-LDH^{P101Q} protein, within 0 hours to 9 hours of incubation, has enzyme activity slightly higher compared with at the beginning, within 9 hours to 67 hours, has a slight decrease, finally, it is 60.53% of the initial enzyme activity, at this time, this enzyme still maintains a relativel high level of enzyme activity, This indicates that D-LDH^{P101Q} has good thermostability.

For the D-lactate dehydrogenase mutant D-LDH^{P101N} according to the present invention, when at 50° C., thermostability is very good, within the heating period from 0 to 67 hours, its enzyme activity always remains at a relatively high level, the final enzyme activity is still maintained at 100.73% of the initial enzyme activity. This indicates that D-LDH^{P101N} has excellent thermostability.

It can be understood that, during the performance of the enzymatic thermostability comparison experiments, comparing each enzyme with itself at different time points is of biological significance, but comparisons between different enzymes are not of biological significance, because initial enzyme activity of each enzyme is different, at all the other time points, according to the initial enzyme activity of each enzyme, relative enzyme activity is calculated and obtained.

### Example 9: D-Lactic Acid Production by Recombinant Geobacillus thermoglucosidasius with D-LDH^{P101Q} Gene through Fed-Batch Fermentation

The nucleotide sequence encoding the mutant protein D-LDH^{P101Q} (SEQ ID No. 3) was integrated into Geobacillus thermoglucosidasius, thereby obtaining recombinant Geobacillus thermoglucosidasius. The recombinant Geobacillus thermoglucosidasius was incubated in a seed culture medium A for fermentation (components: 5 g L⁻¹ soya peptone, 15 g L⁻¹ peptone, 5 g L⁻¹ NaCl) at 60° C. and 200 rpm overnight, making an OD_{620 nm} value reach 2.0 to 8.0.

An inoculum volume of the fermentation seed may be 0.3% (v/v) to 10% (v/v). In this example, the fermentation seed was inoculated at 10% (v/v) into a fermentation medium A (components: 5 g L⁻¹ yeast extract, 3 g L⁻¹ Na₂HPO₄, 3 g L⁻¹ KH₂PO₄, 1 g L⁻¹ NH₄Cl, 0.48 g L⁻¹ MgSO₄, 0.5 g L⁻¹ NaCl, 0.42 g L⁻¹ citric acid, 0.028 g L⁻¹ FeSO₄·7H₂O, 0.01 g L⁻¹ thiamin, Trace Metal Mix (4.4 mg L⁻¹ NiSO₄·6H₂O, 2.86 mg L⁻¹ H₃BO₃, 1.81 mg L⁻¹ MnCl₂·4H₂O, 0.39 mg L⁻¹ Na₂MoO₄·2H₂O, 0.222 mg L⁻¹ ZnSO₄·7H₂O, 0.079 mg L⁻¹ CuSO₄·5H₂O, 0.049 mg L⁻¹ Co(NO₃)₂-6H₂O), 3.1 mg L⁻¹ biotin, 0.1 g L⁻¹ betaine) for fermentation culture. Fermentation conditions: a 5-L fermenter was used, 25% (w/v) Ca(OH)₂ was used to control a pH at 7.0, a speed of agitation was 80 rpm, a fermentation temperature was 60° C. An initial glucose concentration was 100 g L⁻¹, in the process of fermentation, when the glucose concentration was 0.0 g/L to 40.0 g/L, to the fermenter, glucose was added to maintain the glucose concentration in the range of 0 to 70 g/L, a fermentation period was 48 hours.

The fermentation broth was boiled at 100° C. for 10 minutes, then 2 mL was taken and placed in a 100 mL volumetric flask, and 2 mL of 2M sulfuric acid was used for acid-hydrolysis for 10 minutes, the volume was then brought to 100 mL, finally, the above solution was centrifuged at 8,000 rpm for 10 minutes, and after being filtered through a 0.22-micron syringe membrane filter for aqueous filtration, subjected to high-performance liquid chromatography for detecting D-lactic acid.

According to the results of high-performance liquid chromatography, in the fermentation broth resulting from fermentation for 48 hours, a D-lactic acid titer was 153.07 g L⁻¹, a D-lactic acid yield was 93.04%, a D-lactic acid production rate was 3.189 g L⁻¹ h⁻¹.

### Example 10: D-Lactic Acid Production by Recombinant Bacillus licheniformis with D-LDH^{P101Q} Gene through Fed-Batch Fermentation

The nucleotide sequence encoding the mutant protein D-LDH^{P101Q} (SEQ ID No. 3) was integrated into Bacillus licheniformis, thereby obtaining recombinant Bacillus lichenformis. The recombinant Bacillus lichenformis was incubated in a seed culture medium B for fermentation (components: 100 g L⁻¹glucose, 10 g L⁻¹ yeast extract, 5 g L⁻¹ peptone, 50 g L⁻¹ CaCO₃) at 50° C. without agitation overnight, making an OD_{620 nm} value reach 2.0 to 8.0.

The fermentation seed was inoculated at 10% (v/v) into a fermentation medium B (components: 40 g L⁻¹ peanut meal, 100 g L⁻¹ glucose and 0.3 g L⁻¹ neutral protease) for fermentation culture. Fermentation conditions: a 5-L fermenter was used, 25% (w/v) Ca(OH)₂ was used to control a pH at 7.0, a speed of agitation was 80 rpm, a fermentation temperature was 50° C. An initial glucose concentration was 100 g L⁻¹, in the process of fermentation, when the glucose concentration was 0.0 g/L to 40.0 g/L, to the fermenter, glucose was added to maintain the glucose concentration in the range of 0 to 100 g/L, a fermentation period was 60 hours.

The fermentation broth was boiled at 100° C. for 10 minutes, then 2 mL was taken and placed in a 100 mL volumetric flask, and 2 mL of 2M sulfuric acid was used for acid-hydrolysis for 10 minutes, the volume was then brought to 100 mL, finally, the above solution was centrifuged at 8,000 rpm for 10 minutes, and after being filtered through a 0.22-micron syringe membrane filter for aqueous filtration, subjected to high-performance liquid chromatography for detecting D-lactic acid.

According to the results of high-performance liquid chromatography, in the fermentation broth resulting from fermentation for 60 hours, a D-lactic acid titer was 145.91 g L⁻¹, a D-lactic acid yield was 86.00%, a D-lactic acid production rate was 2.43 g L⁻¹ h⁻¹.

### Example 11: D-Lactic Acid Production by Recombinant Bacillus licheniformis with D-LDH Gene through Fed-Batch Fermentation

The nucleotide sequence encoding the protein D-LDH (SEQ ID No. 78) was integrated into Bacillus lichenformis, thereby obtaining recombinant Bacillus lichenformis. The recombinant Bacillus lichenformis was incubated in a seed culture medium B for fermentation (components: 100 g L⁻¹glucose, 10 g L⁻¹ yeast extract, 5 g L⁻¹ peptone, 50 g L⁻¹ CaCO₃) at 50° C. without agitation overnight, making an OD_{620 nm} value reach 2.0 to 8.0.

The fermentation seed was inoculated at 10% (v/v) into a fermentation medium B (components: 40 g L⁻¹ peanut meal, 100 g L⁻¹ glucose, 0.3 g L⁻¹ neutral protease) for fermentation culture. Fermentation conditions: a 5-L fermenter was used, 25% (w/v) Ca(OH)₂ was used to control a pH at 7.0, a speed of agitation was 80 rpm, a fermentation temperature was 50° C. An initial glucose concentration was 100 g L⁻¹, in the process of fermentation, when the glucose concentration was 0.0 g/L to 40.0 g/L, to the fermenter, glucose was added to maintain the glucose concentration in the range of 0 to 100 g/L, a fermentation period was 60 hours.

The fermentation broth was boiled at 100° C. for 10 minutes, then 2 mL was taken and placed in a 100 mL volumetric flask, and 2 mL of 2M sulfuric acid was used for acid-hydrolysis for 10 minutes, the volume was then brought to 100 mL, finally, the above solution was centrifuged at 8,000 rpm for 10 minutes, and after being filtered through a 0.22-micron syringe membrane filter for aqueous filtration, subjected to high-performance liquid chromatography for detecting D-lactic acid.

According to the results of high-performance liquid chromatography, in the fermentation broth resulting from fermentation for 60 hours, a D-lactic acid titer was 48.05 g L⁻¹, a D-lactic acid yield was 56.00%, a D-lactic acid production rate was 0.80 g L⁻¹ h⁻¹.

The results of D-lactic acid production by the recombinant microorganisms through fed-batch fermentation in Examples 9 to 11 are summarized in Table 6 below.

TABLE 6
D-Lactic Acid Production by Recombinant Microorganisms
through Fed-Batch Fermentation

| Example | Example 9 | Example 10 | Example 11 |
|---|---|---|---|
| Recombinant | Recombinant | Recombinant | Recombinant |
| Microorganism | Geobacillus | Bacillus | Bacillus |
| | thermoglucosidasius | licheniformis with | licheniformis with |
| | with D-LDH^{P101Q} Gene | D-LDH^{P101Q} Gene | D-LDH Gene |
| Carbon Source | glucose | glucose | glucose |
| Culture Temperature | 60 | 50 | 50 |
| (° C.) | | | |
| Fermentation Time | 48 | 60 | 60 |
| (hours) | | | |
| Titer of D-lactic Acid | 153.07 | 145.91 | 48.05 |
| (g L⁻¹) | | | |
| Yield of D-lactic Acid | 93.04 | 86.00 | 56.00 |
| (%) | | | |
| Rate of D-Lactic Acid | 3.189 | 2.43 | 0.80 |
| Production (g L⁻¹ h⁻¹) | | | |

As can be seen from the results of Table 6, when the recombinant Geobacillus thermoglucosidasius with the D-LDH^{P101Q}-coding sequence or the recombinant Bacillus licheniformis with the D-LDH^{P101Q}-coding sequence according to the present invention is used to produce D-lactic acid via fed-batch fermentation, a resulting D-lactic acid titer is up to 145 g L⁻¹ or above, a D-lactic acid yield is up to 86% or above, and a D-lactic acid production rate is up to 3.189 g L⁻¹ h⁻¹.

In contrast, when Bacillus lichenformis with the unmutated D-LDH gene is used in Example 11 to produce D-lactic acid via fed-batch fermentation, under the same fermentation conditions as Example 10 using the recombinant Bacillus lichenformis with the mutant gene D-LDHP^{101Q}, a D-lactic acid titer is only 48.05 g L⁻¹, much lower than a D-lactic acid titer of 145.91 g L⁻¹ in Example 10. At the same time, a D-lactic acid yield in Example 11 is only 56%, a D-lactic acid production rate is only 0.80 g L⁻¹ h⁻¹, both significantly lower than corresponding values in Example 10. This indicates that the D-lactate dehydrogenase mutants according to the present invention significantly improve the titer, yield and production rate in D-lactic acid production through fed-batch fermentation, reflecting significantly improved catalytic performance.

Preferred specific embodiments of the present application are described in detail above. It should be understood that those of ordinary skill in the art can make various modifications and changes based on the concept of the present application without exerting any creative effort. Accordingly, all technical solutions that can be obtained by those skilled in the art through logical analysis, inference or limited experimentation in accordance with the concept of the present invention on the basis of the prior art should fall within the scope as defined by the appended claims.

1. A method for constructing a lactic acid-producing strain, characterized by genetically engineering a starting strain to increase lactic acid production, wherein the engineering comprises: 1) introducing a lactic acid synthesis pathway; 2) optimizing the lactic acid synthesis pathway; and 3) inhibiting by-product synthesis pathways.

2. The method according to claim 1, wherein the starting strain is a lactic acid- and/or pyruvic acid-producing strain; preferably, the starting strain is able to produce lactic acid and/or pyruvic acid at a temperature of 37° C. to 70° C.

3. The method according to claim 2, wherein the starting strain includes microorganisms of genus Bacillus and Geobacillus.

4. The method according to claim 3, wherein the starting strain includes Bacillus lichenformis, Bacillus coagulans, Bacillus smithii, Bacillus pallidus and Geobacillus thermoglucosidasius.

5. The method according to claim 1, wherein introducing the lactic acid synthesis pathway comprises introducing an L-lactate dehydrogenase gene L-ldh or a D-lactate dehydrogenase gene D-ldh into the starting strain.

6. The method according to claim 1, wherein optimizing the lactic acid synthesis pathway comprises introducing, into the starting strain, coding sequences of one or more genes selected from: a lactate dehydrogenase gene ldh, a 6-phosphofructokinase gene pfk and a pyruvate kinase gene pyk, wherein the lactate dehydrogenase gene ldh is an L-lactate dehydrogenase gene L-ldh or a D-lactate dehydrogenase gene D-ldh.

7. The method according to claim 5, wherein the introduction into the starting strain comprises: integration into a genome of the starting strain or expression in a plasmid form in the starting strain; preferably, the introduction comprises introducing single-copy or multi-copy the coding sequences of the genes; preferably, the coding sequences of the genes are introduced as individual single-gene expression fragments or as tandem expression fragments of the coding sequences of the genes.

8. The method according to claim 1, wherein inhibiting the by-product synthesis pathways comprises knocking out or down one or more coding genes in the starting strain selected from: a lactate dehydrogenase gene ldh, a formate acetyltransferase gene pflB, an acetaldehyde dehydrogenase gene acdh, an aldehyde-alcohol dehydrogenase gene aadh and a phosphate acetyltransferase gene pta, wherein the lactate dehydrogenase gene ldh is an L-lactate dehydrogenase gene L-ldh or a D-lactate dehydrogenase gene D-ldh.

9. The method according to claim 1, wherein the lactic acid is L-lactic acid or D-lactic acid.

10. The method according to claim 5, wherein the D-lactate dehydrogenase gene ldh encodes an amino acid sequence as shown in SEQ ID No. 9 or SEQ ID No. 10, or encodes an amino acid sequence exhibiting 30% or higher identity to the amino acid sequence as shown in SEQ ID No. 9 or SEQ ID No. 10.

11. The method according to claim 5, wherein the L-lactate dehydrogenase gene L-ldh is derived from Bacillus coagulans and/or Geobacillus thermoglucosidasius, the D-lactate dehydrogenase gene D-ldh is derived from Bacillus lichenformis, the 6-phosphofructokinase gene pfk is derived from Bacillus coagulans, and the pyruvate kinase gene pyk is derived from Bacillus coagulans.

12. A lactic acid-producing strain, characterized in being constructed in accordance with the method according to claim 1.

13. The strain according to claim 12, wherein the strain is Geobacillus thermoglucosidasius with deposition Nos. CCTCC M 20221822, CCTCC M 20221823, CCTCC M 20221824 and CCTCC M 20221825.

14. Use of the strain according to claim 12 in lactic acid production.

15. The use of claim 14, wherein the lactic acid is L-lactic acid or D-lactic acid.

16. The use of claim 14, wherein the lactic acid has a titer of at least 80 g L⁻¹, and a yield of at least 80%; preferably, the lactic acid has a chiral purity of at least 99.04%.

17. The use of claim 14, wherein a method of the lactic acid production comprises steps of:
1) providing the lactic acid-producing strain according to claim 12;
2) culturing the strain at 37° C. to 70° C. for 6 hours to 15 hours, thereby providing a seed; and
3) subjecting the seed to fermentation culture at 37° C. to 70° C. in a presence of a carbon source, thereby obtaining the lactic acid.

18. The use of claim 17, wherein the carbon source is selected from one or more of glucose, xylose, sucrose, glycerol, arabinose and mannitol; preferably, the lactic acid is L-lactic acid or D-lactic acid.

19. The use of claim 17, wherein in step 3), an inoculum volume of the seed has an OD_{620 nm} value of 0.2 to 0.8; preferably, an initial concentration of the carbon source is 40 g/L to 100 g/L; preferably, the fermentation culture is carried out at a pH value of 6.5 to 7.5 for a period of up to 50 hours; preferably, the fermentation culture is performed with agitation at a speed of 50 rpm to 150 rpm.

20. The use of claim 17, wherein the lactic acid has a titer of at least 80 g L⁻¹ and a yield of at least 80%; preferably, the lactic acid has a chiral purity of at least 99.04%.


SEQUENCE LISTING (81 sequences)

SEQ ID NO: 1 - DNA, 978 bp, Bacillus licheniformis
  source 1..978: mol_type = genomic DNA
atgaaagtaatttttttttctatgcacccgtatgaagaggaatttctgggtccgattctgccgtctgactgggacgtagaaatgaccccggactttctggacgaaaccaccgtggaaaaggctaaaggtgcccaggtagtaagcctgtttgtttctgacaaagctgatggtccggtactggaagcgctgcattcttacggtgtgggcctgctggccctgcgttctgctggctatgatcacatcgatattgagaccgcaaaacgcctgggtatcaaagtagttaacgtgccagcctattctccgcacgctatcgctgaccatactctggctatcatgctggctctgattcgtcgtctgcaccgtgcccatgataaagtgcgcctgggtgattttgatctggatggtctgatgggctttgatctgaacggcaaagttgctggtgtaattggtctgggtaaaatcggtcgcctggtagctacccgcctgaaagcgtttggttgcaaagttctgggctatgatccatacattcagccggaaatcgtagaaaacgttgatctggataccctgatcactcaggctgatatcatttctattcattgtccgctgacccgtgaaaactttcatatgtttaacgaagagacttttaagcgtatgaaaccgggtgctattctggttaacaccgcgcgtggtggtctgatcgataccaaggccctgctggaggccctgaagtctggtaaactgggcggcgcagccctggatgtgtatgaatatgaacgtggcctgttttttaaaaaccaccaaaaagaaggtatcaaagacccgtatctggcccagctgctgggtctggccaacgtagtgctgaccggtcatcaggcctttctgacccgtgaggctgtaaaaaacatcgaagaaactaccgtagaaaacattctggaatggcaaaagaacccgcaggcaaagctgaaaaacgaaatctaa

SEQ ID NO: 2 - DNA, 978 bp, synthetic construct
  source 1..978: mol_type = other DNA
atgaaagtaatttttttttctatgcacccgtatgaagaggaatttctgggtccgattctgccgtctgactgggacgtagaaatgaccccggactttctggacgaaaccaccgtggaaaaggctaaaggtgcccaggtagtaagcctgtttgtttctgacaaagctgatggtccggtactggaagcgctgcattcttacggtgtgggcctgctggccctgcgttctgctggctatgatcacatcgatattgagaccgcaaaacgcctgggtatcaaagtagttaacgtgccagcctattctcagcacgctatcgctgaccatactctggctatcatgctggctctgattcgtcgtctgcaccgtgcccatgataaagtgcgcctgggtgattttgatctggatggtctgatgggctttgatctgaacggcaaagttgctggtgtaattggtctgggtaaaatcggtcgcctggtagctacccgcctgaaagcgtttggttgcaaagttctgggctatgatccatacattcagccggaaatcgtagaaaacgttgatctggataccctgatcactcaggctgatatcatttctattcattgtccgctgacccgtgaaaactttcatatgtttaacgaagagacttttaagcgtatgaaaccgggtgctattctggttaacaccgcgcgtggtggtctgatcgataccaaggccctgctggaggccctgaagtctggtaaactgggcggcgcagccctggatgtgtatgaatatgaacgtggcctgttttttaaaaaccaccaaaaagaaggtatcaaagacccgtatctggcccagctgctgggtctggccaacgtagtgctgaccggtcatcaggcctttctgacccgtgaggctgtaaaaaacatcgaagaaactaccgtagaaaacattctggaatggcaaaagaacccgcaggcaaagctgaaaaacgaaatctaa

SEQ ID NO: 3 - DNA, 978 bp, synthetic construct
  source 1..978: mol_type = other DNA
atgaaagtaattttttttagcatgcatccgtatgaagaagaatttttaggcccgattttaccgtcggattgggatgtagaaatgacgccggattttttagatgaaacgacggtggaaaaagcgaaaggagcgcaagtagtaagcttgtttgtttcggataaagcggatggcccggtacttgaagcgcttcattcgtatggagtgggccttttggcgcttcgcagcgcgggctatgatcatattgatattgaaacagcgaaacgcctgggcattaaagtagttaatgtgccggcgtattcgcaacatgcgattgcggatcatacattagcgattatgcttgcgcttattcgccgccttcatcgcgcgcatgataaagtgcgcctgggagattttgatcttgatggccttatgggctttgatttaaatggcaaagttgcgggcgtaattggccttggcaaaattggccgcctggtagcgacacgcttaaaagcgtttggctgcaaagttttaggctatgatccgtatattcaaccggaaattgtagaaaatgttgatttggatacacttatcacacaagcggatatcatttcgattcattgtccgcttacgcgcgaaaattttcatatgtttaatgaagaaacatttaaacgcatgaaaccgggcgcgattttggttaacacggcgcgcggaggccttatagatacaaaagcgttgcttgaagcgttaaaatcgggaaaacttggcggcgcggcgcttgatgtgtatgaatatgaacgcggccttttttttaaaaaccatcaaaaagaaggcattaaagatccgtatcttgcgcaacttttgggcttggcgaatgtagtgttaacaggccatcaagcgtttcttacgcgcgaagcggtaaaaaacattgaagaaacaacagtagaaaatattttagaatggcaaaaaaatccgcaagcgaaacttaaaaatgaaatctga

SEQ ID NO: 4 - DNA, 978 bp, synthetic construct
  source 1..978: mol_type = other DNA
atgaaagtaatttttttttctatgcacccgtatgaagaggaatttctgggtccgattctgccgtctgactgggacgtagaaatgaccccggactttctggacgaaaccaccgtggaaaaggctaaaggtgcccaggtagtaagcctgtttgtttctgacaaagctgatggtccggtactggaagcgctgcattcttacggtgtgggcctgctggccctgcgttctgctggctatgatcacatcgatattgagaccgcaaaacgcctgggtatcaaagtagttaacgtgccagcctattctaaccacgctatcgctgaccatactctggctatcatgctggctctgattcgtcgtctgcaccgtgcccatgataaagtgcgcctgggtgattttgatctggatggtctgatgggctttgatctgaacggcaaagttgctggtgtaattggtctgggtaaaatcggtcgcctggtagctacccgcctgaaagcgtttggttgcaaagttctgggctatgatccatacattcagccggaaatcgtagaaaacgttgatctggataccctgatcactcaggctgatatcatttctattcattgtccgctgacccgtgaaaactttcatatgtttaacgaagagacttttaagcgtatgaaaccgggtgctattctggttaacaccgcgcgtggtggtctgatcgataccaaggccctgctggaggccctgaagtctggtaaactgggcggcgcagccctggatgtgtatgaatatgaacgtggcctgttttttaaaaaccaccaaaaagaaggtatcaaagacccgtatctggcccagctgctgggtctggccaacgtagtgctgaccggtcatcaggcctttctgacccgtgaggctgtaaaaaacatcgaagaaactaccgtagaaaacattctggaatggcaaaagaacccgcaggcaaagctgaaaaacgaaatctaa

SEQ ID NO: 5 - DNA, 939 bp, Bacillus coagulans
  source 1..939: mol_type = genomic DNA
atgaaaaaagtaaaccgtgttgcagtgatcggaactggcgcagtaggcacaagttattgctatgcaatgattaaccagggtgttgcagaagagcttgttttaatcgatattaacgaagcaaaagcagaaggggaagccatggacctgaaccacggcctgccatttgcgcctacgccgacccgcgtttggaaaggcgattattccgattgcggcactgccgaccttgttgtcattacggcaggttccccgcaaaaaccgggcgaaacaaggcttgatcttgtttccaaaaacgcaaaaatttttaaaggcatgattaagagcatcatggacagcggctttaacgggatttttcttgttgccagcaacccggttgacattttgacatatgtaacttggaaagagtccggcctgccgaaagaacatgttatcggttcgggcacagtgcttgactccgcgcgtctccgcaactctttgagcgcccaatttggaattgacccgcgcaatgtgcatgctgcgattatcggcgaacacggcgatacggaacttccggtatggagccatacaactatcggttacgatacgattgaaagttatctacaaaaaggaattattgacgaaaagacgttagatgacatttttgtcaatacgagagatgcggcttatcatattattgaacgaaaaggggccacattttacggcatcgggatgtccctgacccggattacaagggcaatcctgaacaatgaaaacagcgtattgacggtctctgcatttcttgaaggccaatacggaaacagcgatgtgtacgttggcgttccggccatcatcaatcgccagggcatccgtgaagtggttgaaatcaaactgaacgaaaaagaacaggaacagttcaatcattctgtaaaagtgctaaaagaaacaatggcaccgatattgtaa

SEQ ID NO: 6 - DNA, 2745 bp, Bacillus coagulans
  source 1..2745: mol_type = genomic DNA
atgaagcgaattggagtattgacaagcggcggcgatgcaccggggatgaatgcggcggtccgcgcgattgcccgtaaagggatttatcacggcctggaagtttacggcattcgccaaggttataacggattgattcaaggaaacatccaaaagctcgaagcaggatctgttggcgatattctccagcggggcggcacggttttgcagtcggcaagaagcgaagaattcaaaacgccggaagggcagcaaaaagcgatcaggcagctgaaagaccatggcattgaagcgctcgttgtgatcggcggcgacggttcctaccaaggggccaaaaagttgacggaacagggctttaactgcattggtgtgccagggacaatcgataacgacatcccggggacggattttacaatcggttttgatacggcattgaacacagtgcttgatgcgattgataaaattcgcgacaccgcttcttcccacgaacgcacctttattattgaagtcatgggcagaaatgccggggatatcgcgctctggtccggcctggccggcggagccgaatcgattattattccggaagaaaaatatgacttaaaagatgtcgtggagcgtcttgaacaggggagaaaacgcggcaaacgccacagcatcatcattgtcgcggaaggcgtgatgagcggcaacgagtttgctgaacaattgaaaaaaaccggtgtgatcggcgatacccgcgtttctgttctcggccatatccagcgcggcggttctccgacggcatttgaccgcgtgcttgcaagccgcctcggcgcaagggctgttgaactgctgcttgaaggaaaagggggccgcgctgtcggcattcaaaataaccagctggttgaccacgatatccttgagattctcggaaaaccgcacgccgttaataaaaacatgtacaagctgtcgaaagaattgtcgatctaacgtttcttaggaggaaataaaatgaaaaaaaccaaaattgtatgtacaatcggacctgccagtgaaagtgtggaaatgcttgaaagattaatggcaaacgggatggatgtttgccgcctgaacttctcgcacggcagccatgaggaacatcttgcccggattaaaaatatccgtgaagctgcaaaaaaccaaaacaaaacgatcgggcttctgctcgatacaaagggcccggaaatccgcacccatgatatgaaagacggcggattcgagctcgttgaaggcatgacaccggtcatttcaatgacagaagtgctcgggacaccggaaaaattttcggtcacatatgaagggctgattgatgatgtgcacgttggctctaaaattttacttgatgacggtttgattgaactggaagtgacggccatcgataaaaacgccggtgaaatccatacaaaagtgctgaaccgcggcgttttgaaaaacaaaaaaggtgttaacgtcccgggtgtttccgtgaaccttccgggcatcaccgaaaaagacgtgagcgatatcctgttcgggcttgaacaaggcattgacttcattgcggcttcgtttgtacgccggccgtccgacgttttggaaatccgccagctccttgaagaacacgatgctttgcatgtgaaaattttccctaaaattgaaaaccaggaaggcgtcgacaatatcgatgaaatccttgcggtatcagacggcttaatggttgcccgcggcgacctcggcgttgaaattccgaccgaagcggtgccgctcgtacaaaaagaaatgatcagaaaatgtaatacgctcggcaaaccggtgattaccgcaacgcaaatgcttgattcgatgcaacgcaacccgcgcccgacccgcgcggaagcaagcgacgtggccaacgccatttttgacggcacggatgcgatcatgctttccggcgaaacggcagccgggaaatatcctgctgaagcggttaagacgatgtacaatattgcggttcatgtggaaaaagcaattaaccatcgcgatattctgaacaagcgcagcaagagcacggaccataatatgacagacgctatctgccagtccgttgcccatacggctttaaatcttgatgtgaatgccattattgcgccgactgaaagcggctatacggcacgcatgatctccaaataccgcccggcggccccaatcattgctgtcacgagcgatccgaaagtacaacgcggcttaactgttgtgtccggcgtatacccacaattgggcacaaaggcaaacaatacggatgaaatgcttgaaattgcagtggaggaagcgttgaaatccgaaatcgtccatcacggcgaccttgtgatcattacagcaggcgtcccggttggtgggaaaggcaccaccaacctgatgaaagtgcacctgatcggtgatatattggcaaaaggccagggaatcggcagaaaatcggcattcggcccggtcatcgttgctgaaagccctgaagaagcaaacgcaaaggcaacagaaggttgtgtgctcgtcacgagaacgaccgacaaagaaatcatgccggccattgaaaaatgcgccgcgctgattacggaagaaggcggcttgacaagccatgctgcagttgttggcatcaatgtcggcattccggtcattgttggcgttgaaaaagccgtttccatttttgaagacgggcaggaagttacggtagatgcggcaaccggctcggtttacaacggccatgcgactgtattgtaa

SEQ ID NO: 7 - DNA, 179 bp, Geobacillus stearothermophilus
  source 1..179: mol_type = genomic DNA
gcggccgcactagtgcgggacggggagctgagtgctcccgttgtttgccgcggcgtctgtcatgaaatggacaaacaatagtcaaacaatcgccacaatcgcgcatgcattgcggtgcgcctttcgcgtaaaatatttatatgaaagtgttcgcattatattgaaggaggatgaatgca

SEQ ID NO: 8 - DNA, 960 bp, Geobacillus thermoglucosidasius
  source 1..960: mol_type = genomic DNA
atgaaacaacaaggcatgaatcgagtagcacttataggaacggggttcgttggggccagctatgcatttgcccttatgaaccaaggaatagcagatgagttagtattgattgatgtaaataagaataaggcagagggcgatgtgatggatttaaatcacggaaaagtattcgcgccgaagccgatgaatatttggtttggagattatcaagattgccaagacgccgatttggtggtgatttgtgcaggggctaaccaaaagccgggagaaacaagactggatcttgttgacaaaaatattaatatcttcaaaacgattgtcgattctgtgatgaaatccggatttgatggcgtttttcttgtggcaacgaacccagtggatattttaacgtatgctacttggaaatttagcgggttaccgaaagagcgggtaatcggctcaggaacgattcttgatacagcaagattccgcttcttgctaagtgaatattttcaagtggctccgaccaatgtacatgcgtatattattggcgagcatggggatacagagctgcctgtttggagccatgcggaaattggaagcattccagttgagcaaatattgatgcaaaacgataactatagaaaagaggatttagacaatatctttgttaatgttcgtgatgcggcatatcaaatcattgagaaaaaaggggcaacgtattacggcattgcaatgggattagtccgtatcactcgtgctattttgcacaatgaaaatgccatcttaaccgtttctgctcatttggacggccaatatggcgaacgaaatgtttatattggcgtgcctgccattatcaaccgaaacggtattcgtgaagtgatggaattgacgctaaatgaaacagaacaacaacaattccatcatagtgtaactgtattaaaagacattctttcccgttattttgatgatgtaaaataa

SEQ ID NO: 9 - protein, 325 aa, synthetic construct
MKVIFFSMHPYEEEFLGPILPSDWDVEMTPDFLDETTVEKAKGAQVVSLFVSDKADGPVLEALHSYGVGLLALRSAGYDHIDIETAKRLGIKVVNVPAYSQHAIADHTLAIMLALIRRLHRAHDKVRLGDFDLDGLMGFDLNGKVAGVIGLGKIGRLVATRLKAFGCKVLGYDPYIQPEIVENVDLDTLITQADIISIHCPLTRENFHMFNEETFKRMKPGAILVNTARGGLIDTKALLEALKSGKLGGAALDVYEYERGLFFKNHQKEGIKDPYLAQLLGLANVVLTGHQAFLTREAVKNIEETTVENILEWQKNPQAKLKNEI

SEQ ID NO: 10 - protein, 325 aa, synthetic construct
MKVIFFSMHPYEEEFLGPILPSDWDVEMTPDFLDETTVEKAKGAQVVSLFVSDKADGPVLEALHSYGVGLLALRSAGYDHIDIETAKRLGIKVVNVPAYSNHAIADHTLAIMLALIRRLHRAHDKVRLGDFDLDGLMGFDLNGKVAGVIGLGKIGRLVATRLKAFGCKVLGYDPYIQPEIVENVDLDTLITQADIISIHCPLTRENFHMFNEETFKRMKPGAILVNTARGGLIDTKALLEALKSGKLGGAALDVYEYERGLFFKNHQKEGIKDPYLAQLLGLANVVLTGHQAFLTREAVKNIEETTVENILEWQKNPQAKLKNEI

SEQ ID NO: 11 - DNA, 22 bp, synthetic construct
  source 1..22: mol_type = other DNA
aggaatattcagcaatttgccc

SEQ ID NO: 12 - DNA, 19 bp, synthetic construct
  source 1..19: mol_type = other DNA
cagctggcacgacaggttt

SEQ ID NO: 13 - DNA, 44 bp, synthetic construct
  source 1..44: mol_type = other DNA
tagttagttagcccttagtgactcggcttgcatctttcgctgca

SEQ ID NO: 14 - DNA, 36 bp, synthetic construct
  source 1..36: mol_type = other DNA
aaaaattactttcatcgctgtctgtcatcctttcca

SEQ ID NO: 15 - DNA, 42 bp, synthetic construct
  source 1..42: mol_type = other DNA
ggatgacagacagcgatgaaagtaatttttttttctatgcac

SEQ ID NO: 16 - DNA, 39 bp, synthetic construct
  source 1..39: mol_type = other DNA
tattcaaagtcagtattagatttcgtttttcagctttgc

SEQ ID NO: 17 - DNA, 41 bp, synthetic construct
  source 1..41: mol_type = other DNA
aaaaacgaaatctaatactgactttgaatacaacaaggtga

SEQ ID NO: 18 - DNA, 50 bp, synthetic construct
  source 1..50: mol_type = other DNA
aacagctatgaccatgattacgccattaatacccttccacttatccaaga

SEQ ID NO: 19 - DNA, 20 bp, synthetic construct
  source 1..20: mol_type = other DNA
tccggtgcacagcttgtttc

SEQ ID NO: 20 - DNA, 23 bp, synthetic construct
  source 1..23: mol_type = other DNA
agctgtatttgcatttttctccg

SEQ ID NO: 21 - DNA, 48 bp, synthetic construct
  source 1..48: mol_type = other DNA
tagttagttagcccttagtgactcggcaacgcgtatgctgaatagtaa

SEQ ID NO: 22 - DNA, 39 bp, synthetic construct
  source 1..39: mol_type = other DNA
cactagtgcggccgcccgctatcagcttcttaatgttgt

SEQ ID NO: 23 - DNA, 32 bp, synthetic construct
  source 1..32: mol_type = other DNA
agaagctgatagcgggcggccgcactagtgcg

SEQ ID NO: 24 - DNA, 40 bp, synthetic construct
  source 1..40: mol_type = other DNA
tccaattcgcttcattgcattcatcctccttcaatataat

SEQ ID NO: 25 - DNA, 39 bp, synthetic construct
  source 1..39: mol_type = other DNA
aggaggatgaatgcaatgaagcgaattggagtattgaca

SEQ ID NO: 26 - DNA, 36 bp, synthetic construct
  source 1..36: mol_type = other DNA
ctgatagcaacttccttacaatacagtcgcatggcc

SEQ ID NO: 27 - DNA, 40 bp, synthetic construct
  source 1..40: mol_type = other DNA
gcgactgtattgtaaggaagttgctatcagctttttcttt

SEQ ID NO: 28 - DNA, 50 bp, synthetic construct
  source 1..50: mol_type = other DNA
aacagctatgaccatgattacgccagattcatctcagcggaaactactaa

SEQ ID NO: 29 - DNA, 22 bp, synthetic construct
  source 1..22: mol_type = other DNA
cgatcgacgtttgtgattgacc

SEQ ID NO: 30 - DNA, 21 bp, synthetic construct
  source 1..21: mol_type = other DNA
tatccaatcaatcgctttccg

SEQ ID NO: 31 - DNA, 46 bp, synthetic construct
  source 1..46: mol_type = other DNA
tagttagttagcccttagtgactcgcacttctgttggagcgtttcc

SEQ ID NO: 32 - DNA, 40 bp, synthetic construct
  source 1..40: mol_type = other DNA
aaaggggacaatttcatgaaattcccaaggttaacagata

SEQ ID NO: 33 - DNA, 38 bp, synthetic construct
  source 1..38: mol_type = other DNA
ccttgggaatttcatgaaattgtcccctttcagtctga

SEQ ID NO: 34 - DNA, 44 bp, synthetic construct
  source 1..44: mol_type = other DNA
aacagctatgaccatgattacgccaggcgcctcgaccatgttaa

SEQ ID NO: 35 - DNA, 25 bp, synthetic construct
  source 1..25: mol_type = other DNA
aagaaaactattatacacacccgca

SEQ ID NO: 36 - DNA, 23 bp, synthetic construct
  source 1..23: mol_type = other DNA
catggtgaggcctatcagtttac

SEQ ID NO: 37 - DNA, 43 bp, synthetic construct
  source 1..43: mol_type = other DNA
tagttagttagcccttagtgactcggcgcagactgccgtcaat

SEQ ID NO: 38 - DNA, 33 bp, synthetic construct
  source 1..33: mol_type = other DNA
ggaaggggaatggactcgaaacgaaaggagcgg

SEQ ID NO: 39 - DNA, 37 bp, synthetic construct
  source 1..37: mol_type = other DNA
ctcctttcgtttcgagtccattccccttcccatataa

SEQ ID NO: 40 - DNA, 46 bp, synthetic construct
  source 1..46: mol_type = other DNA
aacagctatgaccatgattacgccaccatcgttggcaaaaaaattg

SEQ ID NO: 41 - DNA, 25 bp, synthetic construct
  source 1..25: mol_type = other DNA
cttaacacctgtttcatatccaagg

SEQ ID NO: 42 - DNA, 21 bp, synthetic construct
  source 1..21: mol_type = other DNA
gaacattacagaaatcgccgc

SEQ ID NO: 43 - DNA, 49 bp, synthetic construct
  source 1..49: mol_type = other DNA
tagttagttagcccttagtgactcgcaagctatatgctcaagacatcga

SEQ ID NO: 44 - DNA, 36 bp, synthetic construct
  source 1..36: mol_type = other DNA
gagtggtttttatttacgcattctccctcctgattg

SEQ ID NO: 45 - DNA, 37 bp, synthetic construct
  source 1..37: mol_type = other DNA
ggagggagaatgcgtaaataaaaaccactcccccaaa

SEQ ID NO: 46 - DNA, 45 bp, synthetic construct
  source 1..45: mol_type = other DNA
aacagctatgaccatgattacgccacgtttatcaacgcttccaat

SEQ ID NO: 47 - DNA, 21 bp, synthetic construct
  source 1..21: mol_type = other DNA
cgacaaaacagattatcgccc

SEQ ID NO: 48 - DNA, 19 bp, synthetic construct
  source 1..19: mol_type = other DNA
aacgccgatacgtgggaaa

SEQ ID NO: 49 - DNA, 36 bp, synthetic construct
  source 1..36: mol_type = other DNA
aaaaattactttcatacgcattctccctcctgattg

SEQ ID NO: 50 - DNA, 41 bp, synthetic construct
  source 1..41: mol_type = other DNA
ggagggagaatgcgtatgaaagtaattttttttagcatgca

SEQ ID NO: 51 - DNA, 40 bp, synthetic construct
  source 1..40: mol_type = other DNA
gagtggtttttattttcagatttcatttttaagtttcgct

SEQ ID NO: 52 - DNA, 37 bp, synthetic construct
  source 1..37: mol_type = other DNA
aaaaatgaaatctgaaaataaaaaccactcccccaaa

SEQ ID NO: 53 - DNA, 42 bp, synthetic construct
  source 1..42: mol_type = other DNA
tagttagttagcccttagtgactcgaattccaggcgtcgccc

SEQ ID NO: 54 - DNA, 36 bp, synthetic construct
  source 1..36: mol_type = other DNA
aaaaattactttcataacagtttccctcccatgcat

SEQ ID NO: 55 - DNA, 41 bp, synthetic construct
  source 1..41: mol_type = other DNA
gggagggaaactgttatgaaagtaattttttttagcatgca

SEQ ID NO: 56 - DNA, 40 bp, synthetic construct
  source 1..40: mol_type = other DNA
ggaggggggagattatcagatttcatttttaagtttcgct

SEQ ID NO: 57 - DNA, 38 bp, synthetic construct
  source 1..38: mol_type = other DNA
aaaaatgaaatctgataatctcccccctccttcttaag

SEQ ID NO: 58 - DNA, 50 bp, synthetic construct
  source 1..50: mol_type = other DNA
aacagctatgaccatgattacgccacattctttaatgtgcgaataaaagc

SEQ ID NO: 59 - DNA, 20 bp, synthetic construct
  source 1..20: mol_type = other DNA
ttaacggccgctttgtcttc

SEQ ID NO: 60 - DNA, 21 bp, synthetic construct
  source 1..21: mol_type = other DNA
gctttcttccgaaggaggctc

SEQ ID NO: 61 - DNA, 48 bp, synthetic construct
  source 1..48: mol_type = other DNA
tagttagttagcccttagtgactcgtagaagcaaacgtgtttcgtttt

SEQ ID NO: 62 - DNA, 38 bp, synthetic construct
  source 1..38: mol_type = other DNA
tcacctttttctgaagaacgaatcctccctaatgtttg

SEQ ID NO: 63 - DNA, 37 bp, synthetic construct
  source 1..37: mol_type = other DNA
agggaggattcgttcttcagaaaaaggtgacgaaacg

SEQ ID NO: 64 - DNA, 46 bp, synthetic construct
  source 1..46: mol_type = other DNA
aacagctatgaccatgattacgccaagccgctgccgtttatacata

SEQ ID NO: 65 - DNA, 22 bp, synthetic construct
  source 1..22: mol_type = other DNA
gcatatttccgtcctgtcatgc

SEQ ID NO: 66 - DNA, 21 bp, synthetic construct
  source 1..21: mol_type = other DNA
ctcggaacaatcgtcgggtat

SEQ ID NO: 67 - DNA, 36 bp, synthetic construct
  source 1..36: mol_type = other DNA
gtttacttttttcatacgcattctccctcctgattg

SEQ ID NO: 68 - DNA, 39 bp, synthetic construct
  source 1..39: mol_type = other DNA
ggagggagaatgcgtatgaaaaaagtaaaccgtgttgca

SEQ ID NO: 69 - DNA, 39 bp, synthetic construct
  source 1..39: mol_type = other DNA
gagtggtttttatttttacaatatcggtgccattgtttc

SEQ ID NO: 70 - DNA, 37 bp, synthetic construct
  source 1..37: mol_type = other DNA
gcaccgatattgtaaaaataaaaaccactcccccaaa

SEQ ID NO: 71 - protein, 325 aa, synthetic construct
MKVIFFSMHPYEEEFLGPILPSDWDVEMTPDFLDETTVEKAKGAQVVSLFVSDKADGPVLEALHSYGVGLLALRSAGYDHIDIETAKRLGIKVVNVPAYSGHAIADHTLAIMLALIRRLHRAHDKVRLGDFDLDGLMGFDLNGKVAGVIGLGKIGRLVATRLKAFGCKVLGYDPYIQPEIVENVDLDTLITQADIISIHCPLTRENFHMFNEETFKRMKPGAILVNTARGGLIDTKALLEALKSGKLGGAALDVYEYERGLFFKNHQKEGIKDPYLAQLLGLANVVLTGHQAFLTREAVKNIEETTVENILEWQKNPQAKLKNEI

SEQ ID NO: 72 - protein, 325 aa, synthetic construct
MKVIFFSMHPYEEEFLGPILPSDWDVEMTPDFLDETTVEKAKGAQVVSLFVSDKADGPVLEALHSYGVGLLALRSAGYDHIDIETAKRLGIKVVNVPAYSPHAIADHTLAIMLALIRRLHRAHDKVRLGDFDLDGLMGFDLNGKVAGVIGLGKIGRLVATRLKAFGCKVLGYDPYIQPEIVENVDLDTLITQADIISIHCPLTRENFHMFNEETFKRMKPGAILVNTARGGLIDTKALLEALKSGKLGGAALDVYEYERGLFFKNHQKEGIKDPYLAQLLGLANVVLTGHQAFLTREAVKNIEETTVENILEWQKNPQAKLKNEI

SEQ ID NO: 73 - DNA, 978 bp, synthetic construct; identical to SEQ ID NO: 1
  source 1..978: mol_type = other DNA

SEQ ID NO: 74 - DNA, 37 bp, synthetic construct
  source 1..37: mol_type = other DNA
cgcggatccgatgaaagtaatttttttttctatgcac

SEQ ID NO: 75 - DNA, 32 bp, synthetic construct
  source 1..32: mol_type = other DNA
cccaagcttttagatttcgtttttcagctttg

SEQ ID NO: 76 - DNA, 18 bp, synthetic construct
  source 1..18: mol_type = other DNA
tagcgtgctgagaatagg

SEQ ID NO: 77 - DNA, 18 bp, synthetic construct
  source 1..18: mol_type = other DNA
cctattctcagcacgcta

SEQ ID NO: 78 - DNA, 18 bp, synthetic construct
  source 1..18: mol_type = other DNA
tagcgtgcccagaatagg

SEQ ID NO: 79 - DNA, 18 bp, synthetic construct
  source 1..18: mol_type = other DNA
cctattctgggcacgcta

SEQ ID NO: 80 - DNA, 21 bp, synthetic construct
  source 1..21: mol_type = other DNA
tagcgtggttagaataggctg

SEQ ID NO: 81 - DNA, 21 bp, synthetic construct
  source 1..21: mol_type = other DNA
cagcctattctaaccacgcta